(19) United States
(12) Patent Application Publication
(10) Pub. No.: US 2024/0312570 A1
(43) Pub. Date: Sep. 19, 2024

(54) METHOD FOR DETERMINING GUT MICROBIOTA STATUS

(71) Applicant: SOCIETE DES PRODUITS NESTLE S.A., Vevey (CH)

(72) Inventors: SHAILLAY KUMAR DOGRA, Epalinges (CH); NORBERT SPRENGER, Savigny (CH); JELENA BANJAC, Echandens (CH)

(21) Appl. No.: 18/577,134

(22) PCT Filed: Jul. 8, 2022
(86) PCT No.: PCT/EP2022/069026

§ 371 Date: Jan. 5, 2024

(30) Foreign Application Priority Data
Jul. 9, 2021 (EP) 21184684.5

(51) Int. Cl. G16B 40/20; G16B 20/00

(52) U.S. Cl. CPC G16B 40/20; G16B 20/00

(57) ABSTRACT

The present invention provides methods for determining the gut microbiota status of a subject given one or more microbial ratios provided from the subject's gut microbiota data. The present invention also provides methods for maintaining or improving the gut microbiota status of a subject.

### FIELD OF THE INVENTION

The present invention relates to methods for determining the gut microbiota status of a subject. The present invention also relates to methods for maintaining or improving the gut microbiota status of a subject.

### BACKGROUND TO THE INVENTION

The microbiota usually refers to the composition of microorganisms in an ecosystem, such as the gut or other body sites of humans or animals. Alterations of gut microbiota are associated with many diseases and conditions such as Irritable Bowel Syndrome, Inflammatory Bowel Disease, allergy, diabetes, cancer, asthma, and obesity. A high gut microbiota diversity is considered as a marker of healthy gut ecosystem (Dogra, S. K., et al., 2020. Frontiers in Microbiology, 11, p.2245)

The gut microbiota changes rapidly under the influence of different factors such as age, dietary changes or medications to name just a few. In particular, the gut microbiota changes rapidly and dramatically in the first years of life (Yatsunenko, T., et al., 2012. Nature, 486(7402), pp.222-227). For infants, certain factors such as birth mode, antibiotics usage and duration of exclusive breast-feeding impact the gut microbiota. Some other factors such as living location, siblings and furry pets can also influence the infant's gut microbiota (Dogra S. K. 2021. The Nest 48).

Importantly, an infant's gut microbiota establishment has lasting consequences on their later health (Dogra S. K., et al. Gut Microbes. 2015;6(5):321-5). For example, low diversity was seen in type-1 diabetes (T1D) infants and preceded the manifestation of T1D (Kostic, A. D., et al., 2015. Cell host & microbe, 17(2), pp.260-273). In another study, higher diversity was associated with decreased risk of allergic sensitization at school-age and higher microbiome maturity too early in life was associated with increased risk of allergic sensitization and asthma (Galazzo, G., et al., 2020. Gastroenterology, 158(6), pp. 1584-1596). Thus, age-appropriate microbiome maturation is important, and it is critical to promote age-appropriate gut microbiome maturation in infants for its relevance in infant's health over life.

Different approaches have been used to study microbiome maturation

However, known approaches have some limitations, such as introducing biases by working with compositional data of microbial relative abundances. When one microbe abundance changes, by definition, the abundance of all the other microbes changes relatively. Therefore, different scenarios, which have markedly different biological significance, could lead to the same observation. For example, the relative abundance of one microbe may increase either as a result of its absolute abundance increasing, or as a result of the total abundance of other microbes decreasing.

Known approaches to study microbiome maturation may also accentuate geographical differences seen in early life microbiome and are not easy to implement in a practical setting. In particular, known approaches may require complex methods to quantify the total microbial load. Next generation sequencing methods, such as 16S or shotgun metagenomics based methods, are complex, costly, have a few week's turn-around time, and require specialized instruments, skills and bioinformatics analysis.

As another limitation, the prior art approaches to study microbiome require a complex experimental protocol involving collection of fecal stool samples from which DNA is extracted, complex sequencing of the extracted DNA followed by bioinformatic analysis. Finally, obtained data usually require extensive statistical efforts to get interpretable results that could allow making recommendations. Therefore, prior art approaches involve complex and costly sequencing methods, requiring specialized instruments, skills and bioinformatic tools to interpret the microbiome data.

Thus, there is a demand for new approaches to study microbiome maturation and to provide personalised recommendations and nutritional interventions to promote age-appropriate gut microbiome maturation. The new approaches could be easily available and rapidly deployable for example by health care professionals at point of care.

### SUMMARY OF THE INVENTION

The inventors have developed an approach for providing a trajectory of the early life microbiome (ELM) development using microbial ratios. The inventors have shown that microbial ratios can be used as an input table in trajectory derivation methods and that they are still able to get a similar trajectory and model statistics.

Using ratios of microbial taxa avoids introducing biases in the compositional data and these approaches can be implemented with simple tests such as PCR-based methods, which are routinely used in clinics and diagnostic labs. Additionally, semi-quantitative detection methods can be used at clinics, diagnostic labs, other Point-of-Cares (POCs), plus at residential homes, such as of the infants and their caretakers.

The inventors have determined that different microbial taxa can be used as the reference (i.e. in the denominator of the ratio calculation) and have developed methods to determine the most suitable references. The suitability of a microbial taxon can be based on the number of crossings the microbial taxon has with all the other microbial taxa in the dataset and/or on other criteria such as modelling performance statistics, availability of primers for qPCR test, and so on.

Further still, the inventors have described how microbial ratios can be adjusted to keep on or bring back a subject to the ELM trajectory. Personalised recommendations and dietary advice may be given to an infant's caretaker(s) to maintain or bring back the said infant on the ELM trajectory.

In one aspect, the present invention provides a method for providing a trained regression model for determining the gut microbiota status of a subject, wherein the method comprises: (a) providing gut microbiota data from a population of healthy subjects; and (b) training a regression model on the gut microbiota data, wherein the age of the healthy subjects at data collection is regressed on one or more microbial ratios provided from the gut microbiota data.

The age of the healthy subjects at data collection may be regressed on a plurality of microbial ratios provided from the gut microbiota data. In some embodiments, the age of the healthy subjects at data collection is regressed on 2 or more microbial ratios, 3 or more microbial ratios, 4 or more microbial ratios, or 5 or more microbial ratios. In some embodiments, the age of the healthy subjects at data collection is regressed on 50 or fewer microbial ratios, 25 or fewer microbial ratios, 10 or fewer microbial ratios, 9 or fewer microbial ratios, or 8 or fewer microbial ratios. In some embodiments, the age of the healthy subjects at data collection is regressed on from 2 to 50 microbial ratios, from 3 to 25 microbial ratios, from 4 to 10 microbial ratios, or from 5 to 8 microbial ratios.

In some embodiments, the method further comprises determining the microbial ratios from the gut microbiota data. Suitably, the microbial ratios are log-transformed, preferably wherein the logarithm base is 2.

The microbial taxa in the microbial ratios may be taxonomically-classified and/or functionally-classified. Suitably, the microbial taxa in the microbial ratios are taxonomically-classified by phylum, class, order, family, genus and/or species. Suitably, the microbial taxa in the microbial ratios are taxonomically-classified by genus. In some embodiments, the microbial ratios are bacterial ratios, preferably wherein the microbial taxa in the microbial ratios comprise one or more bacterial taxa selected from Escherichia, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Bacteroides, Citrobacter, Neisseria, Bifidobacterium, Lachnospira, Dialister, Ruminococcus, Blautia, Streptococcus, Eggerthella, Enterococcus, and Paraprevotella.

The microbial ratios may each have the same microbial taxon as the denominator of the ratio. In some embodiments, the denominator of the ratio is determined by the number of crossings the microbial taxon has with all the other microbial taxa in the dataset and/or on other criteria such as modelling performance statistics, availability or ease of testing, or the subject of interest. In some embodiments, the microbial taxon used as the denominator of the ratio is selected from Escherichia, Bacteroides, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Citrobacter, Neisseria, Bifidobacterium, Lachnospira, Dialister, Ruminococcus, Blautia, Streptococcus, Eggerthella, Enterococcus, and Paraprevotella. Preferably, the denominator of the ratio is Escherichia or Bacteroides.

In some embodiments, the microbial ratios comprise one or more microbial ratio selected from: Roseburia/Escherichia, Faecalibacterium/Escherichia, Sutterella/Escherichia, SMB53/Escherichia, Collinsella/Escherichia, Ruminococcus/Escherichia, Akkermansia/Escherichia, Veillonella/Escherichia, Parabacteroides/Escherichia, Clostridium/Escherichia, Oscillospira/Escherichia, Megasphaera/Escherichia, Fusobacterium/Escherichia, Bacteroides/Escherichia, Citrobacter/Escherichia, Neisseria/Escherichia, and Lachnospira/Escherichia.

In some embodiments, the microbial ratios comprise one or more microbial ratio selected from: Roseburia/Bacteroides, Faecalibacterium/Bacteroides, Clostridium/Bacteroides, Bifidobacterium/Bacteroides, Neisseria/Bacteroides, Akkermansia/Bacteroides, Dialister/Bacteroides, Ruminococcus/Bacteroides, Escherichia/Bacteroides, Blautia/Bacteroides, Streptococcus/Bacteroides, Parabacteroides/Bacteroides, Eggerthella/Bacteroides, Collinsella/Bacteroides, Veillonella/Bacteroides, Paraprevotella/Bacteroides, Sutterella/Bacteroides, Enterococcus/Bacteroides and Megasphaera/Bacteroides.

The trained regression model may be an Early Life Microbiome (ELM) trajectory. The trained regression model may predict the age of a subject given their gut microbiota data. Suitably, the trained regression model relates the age of a healthy subject to their microbiota age, microbiome maturation index, and/or microbiome maturation age, optionally wherein the microbiota age is a microbiota compositional age and/or a microbiota functional age.

The trained regression model may be for infancy and/or early childhood. In some embodiments, the trained regression model is for 0-5 years of age, 0-3 years of age, 0-2 years of age. In some embodiments, the trained regression model is for 0-24 months of age. In some embodiments, the trained regression model is for 0-12 months of age, 6-12 months of age, or 12-24 months of age.

In some embodiments, the method further comprises obtaining the gut microbiota data from the population of healthy subjects. Suitably, the gut microbiota data is obtained from or obtainable from fecal samples. The gut microbiota data may provide the relative abundance and/or absolute abundance for a plurality of microbial taxa, optionally wherein the gut microbiota data provides the relative abundance for a plurality of microbial taxa. In some embodiment, the gut microbiota data is obtained or obtainable by PCR-based detection, semi-quantitative detection methods, cycling temperature capillary electrophoresis, immunological-based methods, or any combination thereof.

The healthy subjects may be infants and/or children. In some embodiments, the healthy subjects are 0-5 years of age, or 0-3 years of age, or 0-2 years of age. In some embodiments, the healthy subjects are 0-24 months of age. In some embodiments, the healthy subjects are 0-12 months of age, 6-12 months of age, or 12-24 months of age. Suitably, the gut microbiota data from a population of healthy subjects comprises at least 40 samples and/or at least 20 healthy subjects.

The regression model may be a tree-based regression model, such as a random forest regression model. In some embodiments, the age of the healthy subjects at sample collection is also regressed on one or more additional features provided from the gut microbiota data.

In one aspect, the present invention provides a trained regression model obtained or obtainable by a method according to the present invention.

In one aspect, the present invention provides a trained regression model for determining the gut microbiota status of a subject given one or more microbial ratios provided from the subject's gut microbiota data. Suitably, the trained regression model is obtained or is obtainable by a method according to the present invention.

In one aspect, the present invention provides a method for predicting the age of a subject, wherein the method comprises: (a) providing a trained regression model by a method according to the present invention, or a trained regression model according to the present invention; (b) providing gut microbiota data from the subject; and (c) predicting the age of the subject given their gut microbiota data and the trained regression model.

In one aspect, the present invention provides a method for determining the gut microbiota status of a subject, wherein the method comprises: (a) providing a trained regression model by a method according to the present invention, or a trained regression model according to the present invention; (b) providing gut microbiota data from the subject; and (c) determining whether the subject is an outlier or not in the trained regression model; wherein the gut microbiota status of the subject is healthy if the subject is not an outlier in the trained regression model, and/or wherein the gut microbiota status of the subject is not healthy if the subject is an outlier in the trained regression model.

In some embodiments, the subject is an outlier based on the standard errors (SE), confidence intervals, prediction intervals, and/or standard deviations in the trained regression model. In some embodiments, the subject is an outlier if their gut microbiota data is −2SE or less or 2SE or more from the trained regression line, if their gut microbiota data falls outside the 95% confidence interval in the trained regression model, if their gut microbiota data falls outside the 95% prediction interval in the trained regression model, and/or if they have a Z-score of −2 or less or 2 or more in the trained regression model. In some embodiments, the subject is an outlier if their gut microbiota data falls outside the 95% prediction interval in the trained regression model.

In one aspect, the present invention provides a method for determining the gut microbiota status of a subject, wherein the method comprises: (a) providing a trained regression model by a method according to the present invention, or a trained regression model according to the present invention, wherein the trained regression model is an ELM trajectory; (b) providing gut microbiota data from the subject; and (c) determining whether the subject is on or off the ELM trajectory; wherein the gut microbiota status of the subject is healthy if the subject is on the ELM trajectory, and/or wherein the gut microbiota status of the subject is not healthy if the subject is off the ELM trajectory. Suitably, the subject is on the ELM trajectory if the subject's gut microbiota data does not differ significantly from the ELM trajectory and/or wherein the subject is off the ELM trajectory if the subject's gut microbiota data differs significantly from the ELM trajectory.

In some embodiments, the subject is determined to be off the ELM trajectory based on the standard errors (SE), confidence intervals, prediction intervals, and/or standard deviations of the ELM trajectory. In some embodiments, the subject is determined to be off the ELM trajectory if their gut microbiota data is −2SE or less or 2SE or more from the ELM trajectory, if their gut microbiota data falls outside the 95% confidence interval of the ELM trajectory, if their gut microbiota data falls outside the 95% prediction interval of the ELM trajectory, and/or if they have a Z-score of −2 or less or 2 or more. In some embodiments, the subject is determined to be off the ELM trajectory if their gut microbiota data falls outside the 95% prediction interval of the ELM trajectory.

In one aspect, the present invention provides a method for determining the gut microbiota status of a subject, wherein the method comprises predicting the age of the subject by a method according to the present invention, and wherein the gut microbiota status of the subject is healthy if the predicted age of the subject does not differ significantly from the actual age of the subject and/or wherein the gut microbiota status of the subject is not healthy if the predicted age of the subject differs significantly from the actual age of the subject.

In some embodiments, the predicted age of the subject differs significantly from the actual age of the subject if the predicted age of the subject if it differs by about 0.5 years or more, by about 0.6 years or more, by about 0.7 years or more, by about 0.8 years or more, by about 0.9 years or more, or by about 1 year or more.

Suitably, in a method for predicting the age of the subject or determining the gut microbiota according to the present invention, the subject's gut microbiota data is obtained or obtainable by PCR-based detection, semi-quantitative detection methods, cycling temperature capillary electrophoresis, immunological-based methods, or any combination thereof. In some embodiments, the subject's gut microbiota data may provide 2 or more microbial ratios, 3 or more microbial ratios, 4 or more microbial ratios, or 5 or more microbial ratios. In some embodiments, the subject's gut microbiota data provide 50 or fewer microbial ratios, 25 or fewer microbial ratios, 10 or fewer microbial ratios, 9 or fewer microbial ratios, 8 or fewer microbial ratios, or 7 or fewer microbial ratios. In some embodiments, the subject's gut microbiota data provides from 2 to 50 microbial ratios, from 3 to 25 microbial ratios, from 4 to 10 microbial ratios, or from 5 to 7 microbial ratios.

In one aspect, the present invention provides a method for maintaining or improving the gut microbiota status of a subject, wherein the method comprises: (a) determining the gut microbiota status of the subject by a method according to the present invention; and (b) adjusting the diet, nutrient intake, and/or lifestyle of the subject to maintain or improve the subject's gut microbiota status. After adjusting the diet, nutrient intake, and/or lifestyle of the subject the gut microbiota status of the subject may be healthy.

The adjusted diet, nutrient intake, and/or lifestyle of the subject may increase the abundance and/or function of favourable microbial taxa and/or may decrease the abundance and/or function of unfavourable microbial taxa.

In one aspect, the present invention provides a method for determining a subject's diet and/or nutrient intake, wherein the method comprises: (a) determining the gut microbiota status of the subject by a method according to the present invention; and (b) determining the diet and/or nutrient intake required to maintain or improve the gut microbiota status of the subject.

Suitably, the subject is administered food and/or supplements to increase the abundance and/or function of favourable microbial taxa and/or to decrease the abundance and/or function of unfavourable microbial taxa. In some embodiments, the food and/or supplements comprise vitamins, such as Riboflavin (Vitamin B2), Retinol (Vitamin A), and Calciferol (Vitamin D), and/or minerals, such as Manganese, Zinc, and Potassium.

In one aspect, the present invention provides a computer program comprising instructions which, when the program is executed by a computer, cause the computer to carry out the method according to the present invention.

In one aspect, the present invention provides a computer program comprising instructions which, when the program is executed by a computer, cause the computer to determine a trained regression model for determining the gut microbiota status of a subject from a population of healthy subjects, given the age of the healthy subjects at data collection and one or more microbial ratios provided from the healthy subject's gut microbiota data.

In one aspect, the present invention provides a computer program comprising instructions which, when the program is executed by a computer, cause the computer to predict the age of a subject or determine the gut microbiota status of a subject, given one or more microbial ratios provided from the subject's gut microbiota data.

In one aspect, the present invention provides a computer program comprising instructions which, when the program is executed by a computer, cause the computer to predict the age of a subject or determine the gut microbiota status of a subject, given a regression model trained on the gut microbiota data from a population of healthy subjects and the subject's gut microbiota data, wherein the regression model was trained by regressing the age of the healthy subjects at data collection on one or more microbial ratios provided from the healthy subject's gut microbiota data. In some embodiments, the trained regression model is a trained regression model according to the present invention.

In one aspect, the present invention provides a computer-readable medium comprising instructions which, when executed by a computer, cause the computer to carry out the method according to the present invention

In one aspect, the present invention provides a computer-readable medium comprising instructions which, when executed by a computer, cause the computer to determine a trained regression model for determining the gut microbiota status of a subject from a population of healthy subjects, given the age of the healthy subjects at data collection and one or more microbial ratios provided from the healthy subject's gut microbiota data.

In one aspect, the present invention provides a computer-readable medium comprising instructions which, when the program is executed by a computer, cause the computer to predict the age of a subject or determine the gut microbiota status of a subject, given one or more microbial ratios provided from the subject's gut microbiota data.

In one aspect, the present invention provides a computer-readable medium comprising instructions which, when executed by a computer, cause the computer to predict the age of a subject or determine the gut microbiota status of a subject, given a regression model trained on the gut microbiota data from a population of healthy subjects and the subject's gut microbiota data, wherein the regression model was trained by regressing the age of the healthy subjects at data collection on one or more microbial ratios provided from the healthy subject's gut microbiota data. In some embodiments, the trained regression model is a trained regression model according to the present invention.

In one aspect, the present invention provides use of one or more microbial ratios provided from a subject's gut microbiota data to predict the age of the subject or to determine the gut microbiota status of the subject.

In one aspect, the present invention provides use of one or more microbial ratios provided from gut microbiota data from a population of healthy subjects to train a regression model.

In one aspect, the present invention provides use of a trained regression model according to the present invention to predict the age of a subject or to determine the gut microbiota status of a subject.

Suitably, the subject of interest is 0-5 years of age, or 0-3 years of age, or 0-2 years of age, more preferably wherein the subject of interest is 0-24 months of age. Suitably, the subject of interest is 0-12 months of age, 6-12 months of age, or 12-24 months of age.

### DESCRIPTION OF DRAWINGS

FIG. 1—Exemplary crossing or interweaving of microbial taxa

(A,B) Bifidobacterium abundance over time (black-color line) crossing that of Bacteroides (white-color line) (A-actual data, B-smoothened curves).

(C,D) Collinsella abundance over time (black-color line) compared to that of Bacteroides (white-color line) (C-actual data, D-smoothened curves).

FIG. 2—Total number of crossings for each microbial taxon

Total number of crossings found for each microbial taxon when compared against all the other microbial taxa.

FIG. 3—Early Life Microbiome (ELM) trajectory derived using ratios of microbial taxa with Escherichia

Derivation of an ELM trajectory using Genus level data and ratios derived using Escherichia in the denominator.

FIG. 4—The important microbial ratios with Escherichia in the Early Life Microbiome trajectory

The important features (key microbial ratios) constituting the microbiota age model are shown in their order of importance from top to bottom.

FIG. 5—The key microbial ratios with Escherichia in the Early Life Microbiome trajectory per time windows

The key microbial ratios per different time windows are shown.

FIG. 6—Early Life Microbiome trajectory (ELM) derived using ratios of microbial taxa with Bacteroides

Derivation of an ELM trajectory using Genus level data and ratios derived using Bacteroides in the denominator.

FIG. 7—The important microbial ratios with Bacteroides in the Early Life Microbiome trajectory

The important features (key microbial ratios) constituting the microbiota age model are shown in their order of importance from top to bottom.

FIG. 8—The key microbial ratios with Bacteroides in the Early Life Microbiome trajectory per time windows

The key microbial ratios per different time windows are shown.

FIG. 9—off the trajectory sample for Early Life Microbiome (ELM) trajectory derived using microbial ratios with Escherichia

FIG. 10—off the trajectory sample for Early Life Microbiome (ELM) trajectory derived using microbial ratios with Escherichia

FIG. 11—off the trajectory sample for Early Life Microbiome (ELM) trajectory derived using microbial ratios with Bacteroides

FIG. 12—off the trajectory sample for Early Life Microbiome (ELM) trajectory derived using microbial ratios with Bacteroides

FIG. 13—bringing back an off the Early Life Microbiome (ELM) trajectory sample back on to the trajectory

For the off the ELM trajectory example presented in FIG. 9, the simulation exercise led to this sample being back on the ELM trajectory.

FIG. 14—bringing back an off the Early Life Microbiome (ELM) trajectory sample back on to the trajectory

For the off the ELM trajectory example presented in FIG. 10, the simulation exercise led to this sample being back on the ELM trajectory.

FIG. 15—bringing back an off the Early Life Microbiome (ELM) trajectory sample back on to the trajectory

For the off the ELM trajectory example presented in FIG. 11, the simulation exercise led to this sample being back on the ELM trajectory.

FIG. 16—bringing back an off the Early Life Microbiome (ELM) trajectory sample back on to the trajectory

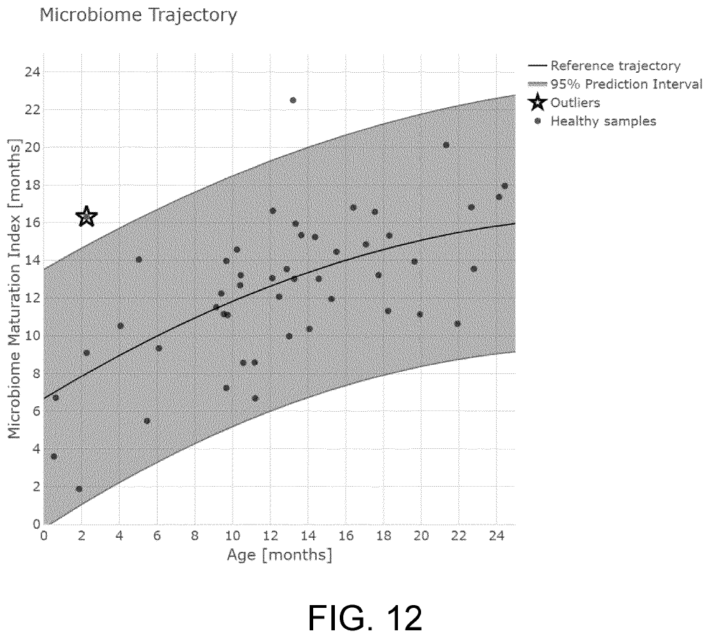

For the off the ELM trajectory example presented in FIG. 12, the simulation exercise led to this sample being back on the ELM trajectory.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by doctors, health-care professionals, lab technicians, infant care providers and so on.

### Method for Providing a Trained Regression Model

The present invention provides a method for providing a trained regression model for determining the gut microbiota status of a subject. The present invention also provides a trained regression model obtained or obtainable by such a method.

### Microbiota and Microbiome

The “gut microbiota” is the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g. Berg, G., et al., 2020. Microbiome, 8(1), pp. 1-22).

In the present invention, the term “gut microbiome” may therefore be used interchangeably with the term “gut microbiota”.

A subject's “gut microbiota status” may refer to the state, condition, or development of a subject's gut microbiota at a particular time. The term “gut microbiota status” may refer to a subject's microbiota age, microbiome maturation index, and/or Early Life Microbiome (ELM) trajectory.

A subject's “microbiota age” can refer to the predicted age of a subject based on their gut microbiota data. For example, the actual/chronological age of subject may be predicted from gut microbiota data obtained from a fecal sample using machine learning based Artificial Intelligence approaches. The term “microbiota age” encompasses both a subject's “microbiota compositional age” and “microbiota functional age”. A “microbiota compositional age” may refer to a microbiota age which is determined using microbial composition data such as at genus level or species level. A “microbiota functional age” may refer to a microbiota age which is determined using functional data such as pathway modules/submodules or metabolites data.

A subject's “microbiome maturation index” or “microbiome maturation age” may be obtained as above, from compositional or functional data, or obtained from compositional and functional data (and other similar data).

A subject's “ELM trajectory” may refer to a fitted curve which is obtained to describe the relation of “microbiota age” or “microbiome maturation index” with actual age. The curve may be fitted by methods such as LOESS or smooth splines using another cohort or subset of data (for external validation purposes).

### Gut Microbiota Data From a Population of Healthy Subjects

The method for providing a trained regression model may comprise providing gut microbiota data from a population of healthy subjects. Suitably, the method further comprises obtaining the gut microbiota data from the population of healthy subjects.

In one aspect the present invention provides a method for providing a trained regression model for determining the gut microbiota status of a subject, wherein the method comprises: (a) obtaining gut microbiota data from a population of healthy subjects; and (b) training a regression model on the gut microbiota data, wherein the age of the healthy subjects at data collection is regressed on one or more microbial ratios provided from the gut microbiota data.

### Gut Microbiota Data

The gut microbiota data may be obtained or obtainable by any suitable sampling method. For example, gut microbiota data may be obtained or obtainable by any method described in Tang, Q., et al., 2020. Frontiers in cellular and infection microbiology, 10, p. 151.

The gut microbiota data may be obtained from or obtainable from fecal samples, endoscopy samples (e.g. biopsy samples, luminal brush samples, laser capture microdissection samples), aspirated intestinal fluid samples, surgery samples, or by in vivo models or intelligent capsule (see e.g. Tang, Q., et al., 2020. Frontiers in cellular and infection microbiology, 10, p. 151).

Suitably, the gut microbiota data may be obtained from or obtainable from fecal samples. Fecal samples are naturally collected, non-invasive and can be sampled repeatedly. Fecal materials instantly frozen at −80° C. that can maintain microbial integrity without preservatives have been widely regarded as the gold standard for gut microbiota profiling, but other storage methods with or without preservatives can also be utilised to achieve microbiota compositions similar to those of fresh samples.

The gut microbiota data may be obtained by or obtainable from the samples by any suitable method. For example, the gut microbiota data may be obtained by or obtainable from the samples by sequencing methods (e.g. next-generation sequencing (NGS) methods), PCR-based methods, semi-quantitative detection methods (e.g. from SwissDeCode), cycling temperature capillary electrophoresis (e.g. from REM analytics), immunological-based methods, cell-based methods, or any combination thereof.

In some embodiments, the gut microbiota data is obtained by or obtainable by sequencing methods (e.g. next-generation sequencing (NGS) methods). NGS enables the profiling of the genomic DNA of all the microorganisms present in a sample. NGS methods can include targeted (e.g. 16S ribosomal RNA sequencing) and/or shotgun sequencing approaches, e.g. as described in Poussin, C., et al., 2018. Drug discovery today, 23(9), pp. 1644-1657.

In some embodiments, the gut microbiota data is obtained or obtainable by PCR-based methods. For example, the gut microbiota data may be obtained by or obtainable by PCR, multiplex PCR (mPCR), and/or quantitative PCR (qPCR). Suitably, the gut microbiota data may be obtained by or obtainable by qPCR, e.g. as described in Jian, C., et al., 2020. PLOS One, 15(1), p.e0227285.

In some embodiments, the gut microbiota data is obtained by or obtainable by semi-quantitative detection methods. For example, the gut microbiota data may be obtained by or obtainable by culture method, denaturing gradient gel electrophoresis (DGGE), terminal restriction fragment length polymorphism (T-RFLP), fluorescence in situ hybridization (FISH), and/or DNA microarrays, e.g. as described in Fraher, M. H., et al., 2012. Nature reviews Gastroenterology & hepatology, 9(6), p.312.

In some embodiments, the gut microbiota data is obtained by or obtainable by cycling temperature capillary electrophoresis, e.g. as described in Refinetti, P., et al., 2016. Mitochondrion, 29, pp.65-74.

In some embodiments, the gut microbiota data may be obtained by or obtainable by immunological-based methods. Immunological-based methods may be based on antibody-antigen interactions, whereby a particular antibody will bind to its specific antigen and can use polyclonal or monoclonal antibodies. Enzyme-linked immunosorbent assay (ELISA) and lateral flow immunoassay are among the immunological-based methods which can be used, e.g. as described in Law, J. W. F., et al., 2015. Frontiers in microbiology, 5, p.770. Exemplary methods are described in Amrouche, T., et al., 2006. Journal of microbiological methods, 65(1), pp. 159-170; and Qian, H., et al., 2008. Applied and environmental microbiology, 74(3), pp.833-839.

In some embodiments, the gut microbiota data is obtained by or obtainable by cell-based methods. For example, the gut microbiota data may be obtained by or obtainable by counting microbial cells using flow cytometry, e.g. as described in Galazzo, G., et al., 2020. Frontiers in cellular and infection microbiology, 10, p.403.

In some embodiments, the gut microbiota data is obtained by or obtainable by a combination of one or more methods described above, e.g. as described in Allaband, C., et al., 2019. Clinical Gastroenterology and Hepatology, 17(2), pp.218-230.

The gut microbiota may provide the abundance for a plurality of microbial taxa. For example, the gut microbiota data may provide the abundance for 2 or more (e.g. 2, 3, 4, 5, 6, 7, 8, 9, 10) microbial taxa, 3 or more microbial taxa, 4 or more microbial taxa, 5 or more microbial taxa, 6 or more microbial taxa, 7 or more microbial taxa, 8 or more microbial taxa, 9 or more microbial taxa, or 10 or more microbial taxa. For example, the gut microbiota data may provide the abundance for 100 or fewer microbial taxa, 50 or fewer microbial taxa, 25 or fewer microbial taxa, 10 or fewer microbial taxa, 9 or fewer microbial taxa, or 8 or fewer microbial taxa. For example, the gut microbiota data may provide the abundance for from 2 to 100 microbial taxa, from 3 to 50 microbial taxa, from 4 to 25 microbial taxa, from 5 to 10 microbial taxa, or from 6 to 8 microbial taxa.

The gut microbiota data may provide the relative abundance and/or absolute abundance for the plurality of microbial taxa. Suitably, the gut microbiota data provides the relative abundance for the plurality of microbial taxa.

The microbial taxa may be classified according to any suitable classification, see e.g. Pitt, T. L. and Barer, M. R., 2012. Medical Microbiology, p.24. The microbial taxa may be classified by the same classification system(s) or by one or more different classification systems. The microbial taxa may be taxonomically-classified and/or functionally-classified, as described in more detail below.

The gut microbiota data from a population of healthy subjects may provide 2 or more (e.g. 2, 3, 4, 5, 6, 7, 8, 9, 10) microbial ratios, 3 or more microbial ratios, 4 or more microbial ratios, 5 or more microbial ratios, 6 or more microbial ratios, 7 or more microbial ratios, 8 or more microbial ratios, 9 or more microbial ratios, or 10 or more microbial ratios. Suitably, the gut microbiota data from a population of healthy subjects may provide 100 or fewer microbial ratios, 50 or fewer microbial ratios, 25 or fewer microbial ratios, 10 or fewer microbial ratios, 9 or fewer microbial ratios, 8 or fewer microbial ratios, or 7 or fewer microbial ratios. Suitably, the gut microbiota data from a population of healthy subjects may provide from 2 to 100 microbial ratios, from 2 to 50 microbial ratios, from 3 to 25 microbial ratios, from 4 to 10 microbial ratios, or from 5 to 7 microbial ratios.

The gut microbiota data from a population of healthy subjects may comprise any number of samples suitable for training a regression model. The gut microbiota data from a population of healthy subjects may comprise at least 20 samples, at least 30 samples, at least 40 samples, at least 50 samples, or at least 100 samples. For example, the gut microbiota data from a population of healthy subjects may comprise at least 40 samples. Suitably, the gut microbiota data from a population of healthy subjects may comprise 1000 samples or less, 500 samples or less, or 100 samples or less. Suitably, the gut microbiota data from a population of healthy subjects may comprise from 20 to 1000 samples.

### Population of Healthy Subjects

The “population of healthy subjects” may refer to a population of subjects with no known underlying health conditions. In some embodiments, the population of subjects have no underlying health conditions. The healthy subjects may be human subjects. The subjects may be male and/or female.

The healthy subjects may be of any age. Suitably, the healthy subjects may be infants, toddlers and/or children. The term “infant” may refer to a subject aged from 0 years to 1 year, or from 0 months to less than 1 year. The term “toddler” may refer to a subject aged from 1 year to 3 years, or from 1 year to less than 3 years. The term “child” may refer to a subject aged under 18 years. The healthy subjects may be infants, toddler and/or young children. The term “young child” may refer to a subject aged from 3 years to 5 years, or from 3 years to less than 5 years.

Suitably, the healthy subjects are 5 years of age or less, 4 years of age or less, 3 years of age or less, 2 years of age or less, 1 year of age or less, or 0.5 years of age or less. Suitably, the healthy subjects are 60 months of age or less, 48 months of age or less, 36 months of age or less, 24 months of age or less, 12 months of age or less, or 6 months of age or less.

Suitably, the healthy subjects are 0 years of age or more, 0.5 years of age or more, or 1 year of age or more. Suitably, the healthy subjects are 0 months of age or more, 6 months of age or more, or 12 months of age or more.

Suitably, the healthy subjects are from 0 years to 5 years of age, from 0 years to 4 years of age, from 0 years to 3 years of age, from 0 years to 2 years of age, or from 0 years to 1 year of age. In some embodiments, the healthy subjects are from 0 years to 2 years of age. Suitably, the healthy subjects are from 0 months to 60 months of age, from 0 months to 48 months of age, from 0 months to 36 months of age, from 0 months to 24 months of age, or from 0 months to 12 months of age. In some embodiments, the healthy subjects are from 0 months to 24 months of age.

Suitably, the healthy subjects are from 0 years to 1 year of age, 0.5 years to 1 year of age, or 1 year to 2 years of age. Suitably, the healthy subjects are from 0 months to 12 months of age, 6 months to 12 months of age, or 12 months to 24 months of age.

The population of healthy subjects may comprise any number of subjects suitable for training a regression model. The population of healthy subjects may comprise at least 10 subjects, at least 20 subjects, at least 30 subjects, at least 40 subjects, or at least 50 subjects. For example, the population of healthy subjects may comprise at least 20 subjects. Suitably, the population of healthy subjects may comprise 500 subjects or less, 100 subjects or less, or 50 subjects or less. Suitably, the population of healthy subjects may comprise from 10 to 500 subjects.

The population of healthy subjects may comprise any number of samples from any number of subjects which is suitable for training a regression model. Suitably, the gut microbiota data from a population of healthy subjects may comprise at least 20 samples from at least 10 subjects or at least 40 samples from at least 20 subjects. Suitably, the gut microbiota data from a population of healthy subjects may comprise 1000 samples or less from 500 subjects or less. Suitably, the gut microbiota data from a population of healthy subjects may comprise from 20 to 1000 samples from 10 to 500 subjects, or from 40 to 1000 samples from 20 to 500 subjects.

### Microbial Ratios

The present invention uses one or more microbial ratios to determine the gut microbiota status of a subject.

For example, the method for providing a trained regression model may comprise training a regression model on gut microbiota data from a population of healthy subjects, wherein the age of the healthy subjects at data collection is regressed on one or more microbial ratios provided from the gut microbiota data.

The present invention provides use of one or more microbial ratios provided from gut microbiota data from a population of healthy subjects to train a regression model

As used herein, a microbial ratio refers to a ratio of the abundance of one microbial taxon to the abundance of another microbial taxon and can be represented by the formula:

$$\frac{A b u n dance of first microbial taxon ( numerator of the ratio )}{\begin{matrix}Abun dance of second microbial \\ taxon ( denominator of the ratio )\end{matrix}}$$

The abundance may be a relative or an absolute abundance.

The microbial ratios may be transformed in any way suitable for training a regression model. For example, the microbial ratios may log-transformed and can be represented by the formula:

$$log \frac{Abundance of first microbial taxon}{Abundance of second microbial taxon}$$

The logarithm base can be any suitable logarithm base, for example 2, 3, 4, 5, 6, 7, 8, 9, 10. Suitably, the logarithm base is 2, Euler's number or 10. Suitably, the logarithm base is 2.

The microbial taxa in the microbial ratios may be classified according to any suitable classification, see e.g. Pitt, T. L. and Barer, M. R., 2012. Medical Microbiology, p.24. The microbial taxa may be classified by the same classification system(s) or by one or more different classification systems. The microbial taxa may be taxonomically-classified and/or functionally-classified.

In some embodiments, the microbial taxa are taxonomically-classified. Microbial taxonomy refers to the rank-based classification of microbes. In the scientific classification established by Carl Linnaeus, each species has to be assigned to a genus, which in turn is a lower level of a hierarchy of ranks (family, suborder, order, subclass, class, division/phyla, kingdom and domain). Prokaryotic taxa which have been correctly described are reviewed in e.g. Bergey's manual of Systematic Bacteriology.

Suitably, the microbial taxa in the microbial ratios are taxonomically-classified by phylum, class, order, family, genus and/or species. Suitably, the microbial taxa in the microbial ratios are taxonomically-classified by phylum, genus and/or species. Suitably, the microbial taxa in the microbial ratios are taxonomically-classified by genus and/or species. In some embodiments, the microbial taxa in the microbial ratios are taxonomically-classified by genus.

In some embodiments, the microbial taxa in the microbial ratios are taxonomically-classified by species.

In some embodiments, the microbial taxa are functionally-classified. For example, the microbial taxa may be classified by one or more phenotypic classification systems (e.g. gram stain, morphology, growth requirements, biochemical reactions, serologic systems, environmental reservoirs etc). In some embodiments, the microbial taxa are classified according to biological or metabolic pathways, protein domains or families, functional modules, complex carbohydrate metabolism, antibiotic resistance, virulence factors, bacterial drug targets and endotoxins, mobile genetic elements, and/or any other functional properties, such as those described in Kultima, J. R., et al., 2016. Bioinformatics, 32(16), pp.2520-2523 and Overbeek, R., et al., 2014. Nucleic acids research, 42(D1), pp.D206-D214.

Suitably, the microbial taxa are bacterial taxa (i.e. the microbial ratios are bacterial ratios). Any suitable bacterial taxa may be used, see e.g. Rinninella, E., et al., 2019. Microorganisms, 7(1), p.14.

For example, (e.g. if the microbial ratios are taxonomically-classified by phylum) the microbial taxa may comprise one or more bacterial taxa selected from Actinobacteria, Firmicutes, Bacteroidetes, Proteobacteria, Fusobacteria, and Verrucomicrobia.

For example, (e.g. if the microbial ratios are taxonomically-classified by class) the microbial taxa may comprise one or more bacterial taxa selected from Actinobacteria, Coriobacteriia, Clostridia, Negativicutes, Bacilli, Sphingobacteriia, Bacteroidia, Gamma proteobacteria, Delta proteobacteria, Epsilon proteobacteria, Fusobacteriia, and Verrucomicrobiae.

For example, (e.g. if the microbial ratios are taxonomically-classified by order) the microbial taxa may comprise one or more bacterial taxa selected from Actinomycetales, Bifidobacteriales, Coriobacteriales, Clostiridiales, Veillonellales, Lactobaccillales, Bacillales, Sphingobacteriales, Bacteroidales, Enterobacterales, Desulfovibrionales, Campylobacterales, Fusobacteriales, and Verrucomicrobiales.

For example, (e.g. if the microbial ratios are taxonomically-classified by family) the microbial taxa may comprise one or more bacterial taxa selected from Corynebacteriaceae, Bifidobacteriaceae, Coriobacteriaceae, Clostridiaceae, Lachnospiraceae, Ruminococcaceae, Veillonellaceae, Lactobacilloae, Enterococcaceae, Staphylococcaceae, Sphingobacteriaceae, Bacteroidaceae, Tannerellaceae, Rikenellaceae, Prevotellaceae, Enterobacteriaceae, Desulfovibrionaceae, Helicobacteraceae, Fusobacteriaceae, and Akkermansiaceae.

For example, (e.g. if the microbial ratios are taxonomically-classified by genus) the microbial taxa in the microbial ratios may comprise one or more bacterial taxa selected from Escherichia, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Bacteroides, Citrobacter, Neisseria, Bifidobacterium, Lachnospira, Dialister, Ruminococcus, Blautia, Streptococcus, Eggerthella, Paraprevotella, Corynebacterium, Atopobium, Lactobacillus, Enterococcus, Staphylococcus, Sphingobacterium, Tannerella, Alistipes, Prevotella, Shigella, Desulfovibrio, Bilophila, and Helicobacter.

For example, (e.g. if the microbial ratios are taxonomically-classified by species) the microbial taxa in the microbial ratios may comprise one or more bacterial taxa selected from Bifidobacterium longum, Bifidobacterium bifidum, Faecalibacterium prausnitzii, Clostridium spp., Roseburia intestinalis, Ruminococcus faecis, Dialister invisus, Lactobacillus reuteri, Enterococcus faecium, Staphylococcus leei, Bacteroides fragilis, Bacteroides vulgatus, Bacteroides uniformis, Parabacteroides distasonis, Alistipes finegoldii, Prevotella spp., Escherichia coli, Shigella flexneri, Desulfovibrio intestinalis, Helicobacter pylori, Fusobacterium nucleatum, and Akkermansia muciniphilia.

In some embodiments, the microbial taxa in the microbial ratios comprise one or more bacterial taxa selected from Bacteroides, Bifidobacterium, Oscillospira, Parabacteroides, Sutterella, Blautia, Akkermansia, Bilophila, Megasphaera, Streptococcus, Ruminococcus, Dialister, Odoribacter, Colinsella, Faecalibacterium, Escherichia, Clostridium, Eubacterium, Phascolarctobacterium, Roseburia, Megamonas, Lachnospira, Coprococcus, Fusobacterium, Paraprevotella, Succinivibrio, Epulopiscium, Lactobacillus, Prevotella, Enterococcus, Butyricicoccus, Eggerthella, Dorea, Cetobacterium, Staphylococcus, Paenilbacillus, Anaerotruncus, Rothia, Butyricimonas, Neisseria, SMB53, Veillonella, and Citrobacter.

In some embodiments, the microbial taxa in the microbial ratios comprise one or more bacterial taxa selected from Escherichia, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Bacteroides, Citrobacter, Neisseria, Bifidobacterium, Lachnospira, Dialister, Ruminococcus, Blautia, Streptococcus, Eggerthella, Enterococcus, and Paraprevotella.

In some embodiments, the microbial taxa in the microbial ratios comprise one or more bacterial taxa selected from Escherichia, Roseburia, Faecalibacterium, Sutterella, Collinsella, Akkermansia, and Clostridium. In some embodiments, the microbial taxa in the microbial ratios comprise each of Escherichia, Roseburia, Faecalibacterium, Sutterella, Collinsella, Akkermansia, and Clostridium.

In some embodiments, the microbial taxa in the microbial ratios comprise one or more bacterial taxa selected from Bacteroides, Ruminococcus, Bifidobacterium, Clostridium, Akkermansia, Neisseria, Faecalibacterium, Roseburia, Veillonella, and Enterococcus. In some embodiments, the microbial taxa in the microbial ratios comprise each of Bacteroides, Ruminococcus, Bifidobacterium, Clostridium, Akkermansia, Neisseria, Faecalibacterium, Roseburia, Veillonella, and Enterococcus.

Suitable microbial taxa may be determined by any suitable method. For example, the suitability of a microbial taxon can be based on modelling performance statistics, availability or ease of testing, or on the subject of interest.

In some embodiments, the microbial taxa are determined based on modelling performance statistics. For example, the microbial taxa may provide a trained regression model which is sufficiently accurate to determine a subject's microbiota status or may provide the most accurate trained regression model.

In some embodiments, the microbial taxa are determined based on the availability or ease of testing. For example, the microbial taxa may be ones for which primer for PCR-based methods are available, for which antibodies are available for immunological-based methods, and/or for which tests (e.g. semi-quantitative tests) are commercially available.

In some embodiments, the microbial taxa are determined based on the subject of interest. The gut microbiota changes rapidly under the influence of different factors. For example, the microbial taxa may be ones which are suitable based on the subject's age, diet, medication, birth mode, duration of exclusive breast-feeding, living location, ethnicity, siblings, and pets. Suitably, the microbial taxa may be ones which are suitable based on the subject's age and/or living location.

A plurality of microbial ratios may be used to determine the gut microbiota status of the subject. Suitably, the 2 or more (e.g. 2, 3, 4, 5, 6, 7, 8, 9, 10) microbial ratios, 3 or more microbial ratios, 4 or more microbial ratios, 5 or more microbial ratios, 6 or more microbial ratios, 7 or more microbial ratios, 8 or more microbial ratios, 9 or more microbial ratios, 10 or more microbial ratios are used to determine the gut microbiota status of the subject. Suitably, 100 or fewer microbial ratios, 50 or fewer microbial ratios, 25 or fewer microbial ratios, 10 or fewer microbial ratios, 9 or fewer microbial ratios, 8 or fewer microbial ratios, or 7 or fewer microbial ratios are used to determine the gut microbiota status of the subject. Suitably, from 2 to 100 microbial ratios, from 2 to 50 microbial ratios, from 3 to 25 microbial ratios, from 4 to 10 microbial ratios, or from 5 to 7 microbial ratios are used to determine the gut microbiota status of the subject.

Suitably, the age of the healthy subjects at data collection may be regressed on 2 or more (e.g. 2, 3, 4, 5, 6, 7, 8, 9, 10) microbial ratios, 3 or more microbial ratios, 4 or more microbial ratios, 5 or more microbial ratios, 6 or more microbial ratios, 7 or more microbial ratios, 8 or more microbial ratios, 9 or more microbial ratios, 10 or more microbial ratios. Suitably, the age of the healthy subjects at data collection may be regressed on 100 or fewer microbial ratios, 50 or fewer microbial ratios, 25 or fewer microbial ratios, 10 or fewer microbial ratios, 9 or fewer microbial ratios, 8 or fewer microbial ratios, or 7 or fewer microbial ratios. Suitably, the age of the healthy subjects at data collection may be regressed on from 2 to 100 microbial ratios, from 2 to 50 microbial ratios, from 3 to 25 microbial ratios, from 4 to 10 microbial ratios, or from 5 to 7 microbial ratios.

The methods of the present invention may comprise determining the microbial ratios from the gut microbiota data (e.g. from the gut microbiota data from a population of healthy subjects or from the gut microbiota data from a subject of interest). Accordingly, the gut microbiota data may provide the abundance for each of the microbial taxa in the microbial ratios.

### Reference Microbial Taxon

A plurality of microbial ratios may each have the same microbial taxon as the denominator of the ratio. For example, all of the microbial ratios may each have the same microbial taxon as the denominator of the ratio. The microbial taxon which is a denominator of more than one of the microbial ratios may also be referred to as the “reference microbial taxon” and the microbial ratios may be represented by the formula:

$$\frac{A b undance of nth microbial taxon}{A b u n dance of reference microbial taxon}$$

The abundance may be a relative or an absolute abundance.

The reference microbial taxon be any suitable microbial taxon. Suitably, the reference microbial taxon may be selected from (e.g. if the reference microbial taxon is taxonomically-classified by genus) Escherichia, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Bacteroides, Citrobacter, Neisseria, Bifidobacterium, Lachnospira, Dialister, Ruminococcus, Blautia, Streptococcus, Eggerthella, Paraprevotella, Corynebacterium, Atopobium, Lactobacillus, Enterococcus, Staphylococcus, Sphingobacterium, Tannerella, Alistipes, Prevotella, Shigella, Desulfovibrio, Bilophila, and Helicobacter.

Suitably, the reference microbial taxon may be selected from Bacteroides, Bifidobacterium, Oscillospira, Parabacteroides, Sutterella, Blautia, Akkermansia, Bilophila, Megasphaera, Streptococcus, Ruminococcus, Dialister, Odoribacter, Colinsella, Faecalibacterium, Escherichia, Clostridium, Eubacterium, Phascolarctobacterium, Roseburia, Megamonas, Lachnospira, Coprococcus, Fusobacterium, Paraprevotella, Succinivibrio, Epulopiscium, Lactobacillus, Prevotella, Enterococcus, Butyricicoccus, Eggerthella, Dorea, Cetobacterium, Staphylococcus, Paenilbacillus, Anaerotruncus, Rothia, Butyricimonas, Neisseria, SMB53, Veillonella, and Citrobacter

Suitably, the reference microbial taxon may be selected from Escherichia, Bacteroides, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Citrobacter, Neisseria, Bifidobacterium, Lachnospira, Dialister, Ruminococcus, Blautia, Streptococcus, Eggerthella, Enterococcus, and Paraprevotella.

In some embodiments, the reference microbial taxon is Escherichia or Bacteroides. In some embodiments, the reference microbial taxon is Escherichia. In some embodiments, the reference microbial taxon is Bacteroides.

In some embodiments, all of the microbial ratios each have the same reference microbial taxon.

In some embodiments, all of the microbial ratios each have Escherichia or Bacteroides as the reference microbial taxon. In some embodiments, all of the microbial ratios each have Escherichia as the reference microbial taxon. In some embodiments, all of the microbial ratios each have Bacteroides as the reference microbial taxon.

In some embodiments, the microbial ratios comprise or consist of one or more microbial ratio selected from: abundance of Roseburia/abundance of Escherichia; abundance of Faecalibacterium/abundance of Escherichia; abundance of Sutterella/abundance of Escherichia; abundance of SMB53/abundance of Escherichia; abundance of Collinsella/abundance of Escherichia; abundance of Ruminococcus/abundance of Escherichia; abundance of Akkermansia/abundance of Escherichia; abundance of Veillonella/abundance of Escherichia; abundance of Parabacteroides/abundance of Escherichia; abundance of Clostridium/abundance of Escherichia; abundance of Oscillospira/abundance of Escherichia; abundance of Megasphaera/abundance of Escherichia; abundance of Fusobacterium/abundance of Escherichia; abundance of Bacteroides/abundance of Escherichia; abundance of Citrobacter/abundance of Escherichia; abundance of Neisseria/abundance of Escherichia; and abundance of Lachnospira/abundance of Escherichia. In some embodiments, the microbial ratios comprise or consist of one or more microbial ratio selected from: abundance of Roseburia/abundance of Escherichia; abundance of Faecalibacterium/abundance of Escherichia; abundance of Sutterella/abundance of Escherichia; abundance of Collinsella/abundance of Escherichia; abundance of Akkermansia/abundance of Escherichia; and abundance of Clostridium/abundance of Escherichia. In some embodiments, abundance of the microbial ratios comprise or consist of each of: abundance of Roseburia/abundance of Escherichia; abundance of Faecalibacterium/abundance of Escherichia; abundance of Sutterella/abundance of Escherichia; abundance of Collinsella/abundance of Escherichia; abundance of Akkermansia/abundance of Escherichia; and abundance of Clostridium/abundance of Escherichia.

In some embodiments, the microbial ratios comprise or consist of one or more microbial ratio selected from: abundance of Roseburia/abundance of Bacteroides; abundance of Faecalibacterium/abundance of Bacteroides; abundance of Clostridium/abundance of Bacteroides; abundance of Bifidobacterium/abundance of Bacteroides; abundance of Neisseria/abundance of Bacteroides; abundance of Akkermansia/abundance of Bacteroides; abundance of Dialister/abundance of Bacteroides; abundance of Ruminococcus/abundance of Bacteroides; abundance of Escherichia/abundance of Bacteroides; abundance of Blautia/abundance of Bacteroides; abundance of Streptococcus/abundance of Bacteroides; abundance of Parabacteroides/abundance of Bacteroides; abundance of Eggerthella/abundance of Bacteroides; abundance of Collinsella/abundance of Bacteroides; abundance of Veillonella/abundance of Bacteroides; abundance of Paraprevotella/abundance of Bacteroides; abundance of Sutterella/abundance of Bacteroides; abundance of Enterococcus/abundance of Bacteroides; and abundance of Megasphaera/abundance of Bacteroides. In some embodiments, the microbial ratios comprise or consist of one or more microbial ratio selected from: abundance of Ruminococcus/abundance of Bacteroides; abundance of Bifidobacterium/abundance of Bacteroides; abundance of Clostridium/abundance of Bacteroides; abundance of Akkermansia/abundance of Bacteroides; abundance of Neisseria/abundance of Bacteroides; abundance of Faecalibacterium/abundance of Bacteroides; abundance of Roseburia/abundance of Bacteroides; abundance of Veillonella/abundance of Bacteroides; and abundance of Enterococcus/abundance of Bacteroides. In some embodiments, the microbial ratios comprise each of: abundance of Ruminococcus/abundance of Bacteroides; abundance of Bifidobacterium/abundance of Bacteroides; abundance of Clostridium/abundance of Bacteroides; abundance of Akkermansia/abundance of Bacteroides; abundance of Neisseria/abundance of Bacteroides; abundance of Faecalibacterium/abundance of Bacteroides; abundance of Roseburia/abundance of Bacteroides; abundance of Veillonella/abundance of Bacteroides; and abundance of Enterococcus/abundance of Bacteroides.

The reference microbial taxon may be determined by any suitable method. For example, the suitability of a microbial taxon may be determined by the number of crossings the microbial taxon has with all the other microbial taxa in the dataset and/or on other criteria such as modelling performance statistics, availability or ease of testing, or the subject of interest (as described in detail above).

In some embodiments, the reference microbial taxon is determined by the number of crossings the microbial taxon has with all the other microbial taxa in the healthy population's gut microbiota data. One microbial taxon “crosses” another microbial taxon when one microbial taxon changes from being more abundant than the other microbial taxon to being less abundant than the other microbial taxon (or vice versa). The “number of crossings” is the number of crosses which occur over the course of the gut microbiota data. Suitably, the reference microbial taxon is ranked in the 50^{th} percentile or lower, 40^{th} percentile or lower, 30^{th} percentile or lower, 20^{th} percentile or lower, 10^{th} percentile or lower, 5^{th} percentile or lower based on the number of crossings. Suitably, the reference microbial taxon has the fewest number of crossings.

In some embodiments, the reference microbial taxon is determined based on modelling performance statistics. In some embodiments, the reference microbial taxon is determined based on the availability or ease of testing. In some embodiments, the reference microbial taxon is determined based on the subject of interest.

In some embodiments, the reference microbial taxon is not a microbial taxon which has a high impact on the trained regression model. Any suitable statistical method may be used to identify microbial taxa which have a high impact on the trained regression model, for example based on the feature importance. The feature importance may be determined using any suitable statistical method, for example based on SHapley Additive explanation (SHAP) values (Lundberg S M, et al. Nat Mach Intell. 2020).

In some embodiments, the reference microbial taxon is not Roseburia. In some embodiments, the reference microbial taxon is not Faecalibacterium. In some embodiments, the reference microbial taxon is not Sutterella. In some embodiments, the reference microbial taxon is not Collinsella. In some embodiments, the reference microbial taxon is not Akkermansia. In some embodiments, the reference microbial taxon is not Clostridium. In some embodiments, the reference microbial taxon is not Ruminococcus. In some embodiments, the reference microbial taxon is not Bifidobacterium. In some embodiments, the reference microbial taxon is not Neisseria. In some embodiments, the reference microbial taxon is not Veillonella. In some embodiments, the reference microbial taxon is not Enterococcus.

In some embodiments, the reference microbial taxon is not Roseburia, Faecalibacterium, Sutterella, Collinsella, Akkermansia, or Clostridium. In some embodiments, the reference microbial taxon is not Ruminococcus, Bifidobacterium, Clostridium, Akkermansia, Neisseria, Faecalibacterium, Roseburia, Veillonella, or Enterococcus. In some embodiments, the reference microbial taxon is not Roseburia, Faecalibacterium, Sutterella, Collinsella, Akkermansia, Clostridium, Ruminococcus, Bifidobacterium, Neisseria, Veillonella, or Enterococcus.

### Regression Analysis

The present invention may use regression analysis to relate the age of a population of healthy subjects at microbiota data collection to one or more microbial ratios provided from their microbiota data.

Regression analysis is a set of statistical processes for estimating the relationships between a dependent variable (e.g. the age of the population of healthy subjects at data collection) and one or more independent variables (e.g. one or more microbial ratios from the gut microbiota data). The regression analysis may be used to provide a trained (or fitted) regression model.

The regression analysis may be performed using be any suitable regression model. Suitable regression models will be well known to the skilled person. Exemplary regression models include decision tree regression, linear regression, polynomial regression, quantile regression, ridge regression, lasso regression, elastic net regression, and support vector regression.

Suitably, the regression analysis is performed using machine learning methods. Exemplary machine learning methods include tree-based regression models (e.g. a random forest regression models), recursive partitioning, regularized and shrinkage methods, boosting and gradient descent, and Bayesian methods.

Suitably, the regression model is a tree-based regression model (e.g. a random forest regression model). In some embodiments, the regression model is a random forest regression model.

The regression analysis may be performed by training a regression model on the gut microbiota data. For example, regression analysis may be performed by training a regression model using the age of the healthy subjects at data collection and one or more microbial ratios provided from the gut microbiota data.

As used herein, “training” of “fitting” a regression model may mean determining a function which most closely fits the data according to a suitable statistical criteria. For example, the method of ordinary least squares may be used to compute the function that minimizes the sum of squared differences between the true data and that function.

### Other Additional Features

The present invention may use one or more additional features (in addition to the one or more microbial ratios) to determine the gut microbiota status of a subject.

For example, the method for providing a trained regression model may comprise training a regression model on gut microbiota data from a population of healthy subjects, wherein the age of the healthy subjects at data collection is regressed on one or more microbial ratios provided from the gut microbiota data and one or more additional features.

The term “features” includes responses obtained from the microbiota data and/or the general metadata.

The additional features may include, for example, the relative abundance of one or more microbial taxa. Suitably, the age of the healthy subjects at data collection is regressed on one or more microbial ratios provided from the gut microbiota data and one or more relative abundances provided from the gut microbiota data. Suitably, the age of the healthy subjects at data collection is regressed on 2 or more relative abundances (e.g. 2, 3, 4, 5, 6, 7, 8, 9, 10), 3 or more relative abundances, 4 or more relative abundances, 5 or more relative abundances, 6 or more relative abundances, 7 or more relative abundances, 8 or more relative abundances, 9 or more relative abundances, or 10 or more relative abundances. Suitably, the age of the healthy subjects at data collection is regressed on 100 or fewer relative abundances, 50 or fewer relative abundances, 25 or fewer relative abundances, 10 or fewer relative abundances, 9 or fewer relative abundances, or 8 or fewer relative abundances. Suitably, the age of the healthy subjects at data collection is regressed on from 2 to 100 relative abundances, from 3 to 50 relative abundances, from 4 to 25 relative abundances, or from 5 to 10 relative abundances, or from 6 to 8 relative abundances.

### Trained Regression Model

The present invention provides a trained regression model for determining the gut microbiota status of a subject given one or more microbial ratios provided from the subject's gut microbiota data. The trained regression model may be obtained or obtainable by any method described herein.

The “trained regression model” (also known as a “fitted regression model”) may relate the age of a healthy subject to their microbiota data. The trained regression model may provide a “trained regression function” or “trained regression line”, relating the age of a healthy subject to the one or more microbial ratios described herein, and other statistics such as the standard errors of the regression, confidence intervals, prediction intervals, and/or standard deviations of the regression.

The trained regression model may predict the age of a subject given their gut microbiota data. For example, the trained regression model may predict the age of a subject given one or more microbial ratios described herein. The prediction may be based on assuming the subject is healthy.

The trained regression model may relate the age of a healthy subject to their microbiota age and/or microbiome maturation index. Suitably, the microbiota age is a microbiota compositional age and/or a microbiota functional age.

The trained regression model may be an Early Life Microbiome (ELM) trajectory.

The trained regression model may be for any age. For example, the trained regression model may be for infancy, toddlerhood and/or childhood. The term “infancy” may refer to from 0 years to 1 year of age, or from 0 months to less than 1 year of age. The term “toddlerhood” may refer to from 1 year to 3 years of age, or from 1 year to less than 3 years of age. The term “childhood” may refer to up to 18 years of age. The trained regression model may be for infancy, toddlerhood and/or early childhood. The term “early childhood” may refer to 3 years to 5 years of age, or from 3 years to less than 5 years of age.

Suitably, the trained regression model is for 5 years of age or less, 4 years of age or less, 3 years of age or less, 2 years of age or less, 1 year of age or less, or 0.5 years of age or less. Suitably, the trained regression model is for 60 months of age or less, 48 months of age or less, 36 months of age or less, 24 months of age or less, 12 months of age or less, or 6 months of age or less.

Suitably, the trained regression model is for 0 years of age or more, 0.5 years of age or more, or 1 year of age or more. Suitably, the trained regression model is for 0 months of age or more, 6 months of age or more, or 12 months of age or more.

Suitably, the trained regression model is for from 0 years to 5 years of age, from 0 years to 4 years of age, from 0 years to 3 years of age, from 0 years to 2 years of age, or from 0 years to 1 year of age. In some embodiments, the trained regression model is for from 0 years to 2 years of age. Suitably, the trained regression model is for from 0 months to 60 months of age, from 0 months to 48 months of age, from 0 months to 36 months of age, from 0 months to 24 months of age, or from 0 months to 12 months of age. In some embodiments, the trained regression model is for from 0 months to 24 months of age.

Suitably, the trained regression model is for from 0 years to 1 year of age, 0.5 years to 1 year of age, or 1 year to 2 years of age. Suitably, the trained regression model is for from 0 months to 12 months of age, 6 months to 12 months of age, or 12 months to 24 months of age.

The trained regression model will depend on the population of healthy subjects chosen. The trained regression model may vary, for example, depending on the age, diet, medication, birth mode, duration of exclusive breast-feeding, living location, ethnicity, siblings, and pets of the healthy population.

### Method for Determining the Gut Microbiota Status of a Subject

The present invention provides a method for determining the gut microbiota status of a subject of interest given their gut microbiota data. The method may use any trained regression model described herein.

The present invention also provides use of one or more microbial ratios provided from a subject's gut microbiota data to determine the gut microbiota status of the subject. The present invention also provides use of a trained regression model according to the present invention to determine the gut microbiota status of a subject.

### Subject of Interest

The subject of interest may be any suitable subject. For example, the subject of interest may be a subject with the same characteristics as the healthy population of subjects on which the regression model was trained (except wherein the subject may or may not be healthy). For example, the subject of interest may have an age which falls within the range of ages of the healthy population at data collection. The subject of interest may be human. The subject of interest may be male or female.

The subject of interest may be any age. For example, the subject of interest may be an infant, a toddler or a child. The subject of interest may be an infant, a toddler or a young child.

Suitably, the subject of interest is 5 years of age or less, 4 years of age or less, 3 years of age or less, 2 years of age or less, 1 year of age or less, or 0.5 years of age or less. Suitably, the subject of interest is 60 months of age or less, 48 months of age or less, 36 months of age or less, 24 months of age or less, 12 months of age or less, or 6 months of age or less.

Suitably, the subject of interest is 0 years of age or more, 0.5 years of age or more, or 1 year of age or more. Suitably, the subject of interest is 0 months of age or more, 6 months of age or more, or 12 months of age or more.

Suitably, the subject of interest is from 0 years to 5 years of age, from 0 years to 4 years of age, from 0 years to 3 years of age, from 0 years to 2 years of age, or from 0 years to 1 year of age. In some embodiments, the subject of interest is from 0 years to 2 years of age. Suitably, the subject of interest is from 0 months to 60 months of age, from 0 months to 48 months of age, from 0 months to 36 months of age, from 0 months to 24 months of age, or from 0 months to 12 months of age. In some embodiments, the subject of interest is from 0 months to 24 months of age.

Suitably, subject of interest is from 0 years to 1 year of age, 0.5 years to 1 year of age, or 1 year to 2 years of age. Suitably, the subject of interest is from 0 months to 12 months of age, 6 months to 12 months of age, or 12 months to 24 months of age.

### Gut Microbiota Data From a Subject

The subject's gut microbiota data may be obtained or obtainable by any suitable sampling method described herein. The subject's gut microbiota data may be obtained or obtainable by the same method as the gut microbiota data from the population of healthy subjects or by a different method.

The subject's gut microbiota data may be obtained from or obtainable from a fecal sample, an endoscopy samples (e.g. a biopsy sample, a luminal brush sample, a laser capture microdissection sample), an aspirated intestinal fluid sample, a surgery sample, or by an in vivo model or an intelligent capsule. Preferably, the subject's gut microbiota data is obtained from a fecal sample.

The gut microbiota data may be obtained by or obtainable by any suitable detection method. For example, the gut microbiota data may be obtained by or obtainable by sequencing methods (e.g. next-generation sequencing (NGS) methods), PCR-based methods, semi-quantitative detection methods (e.g. from SwissDeCode), cycling temperature capillary electrophoresis (e.g. from REM analytics), cell-based methods, immunological-based methods, or any combination thereof. Preferably, the gut microbiota data is obtained by or obtainable by PCR-based methods, semi-quantitative detection methods (e.g. from SwissDeCode), cycling temperature capillary electrophoresis (e.g. from REM analytics), or immunological-based methods, or any combination thereof.

One advantage of the present invention is that next generation sequencing methods may not be required to determine a subject's gut microbiota status. Next generation sequencing methods such as 16S or shotgun metagenomics based methods, are complex, costly, have a long turn-around time, and require specialized instruments, skills and bioinformatics analysis. Instead, this can be done with simple tests such as PCR-based, which are routinely used in clinics and diagnostic labs. Additionally, semi-quantitative detection methods can be used at clinics, diagnostic labs, other Point-of-Cares (POCs), plus at residential homes such as of the infants and their caretakers. Also, Cycling Temperature Capillary Electrophoresis can be used to measure ratios and can be used to test multiple ratios in parallel. Further, immunological-based tests can be used.

The subject's gut microbiota data may provide the abundance for a plurality of microbial taxa. For example, the gut microbiota data may provide the abundance for 2 or more (e.g. 2, 3, 4, 5, 6, 7, 8, 9, 10) microbial taxa, 3 or more microbial taxa, 4 or more microbial taxa, 5 or more microbial taxa, 6 or more microbial taxa, 7 or more microbial taxa, 8 or more microbial taxa, 9 or more microbial taxa, or 10 or more microbial taxa. For example, the gut microbiota data may provide the abundance for 100 or fewer, 50 or fewer microbial taxa, 25 or fewer microbial taxa, 10 or fewer microbial taxa, 9 or fewer microbial taxa, or 8 or fewer microbial taxa. For example, the gut microbiota data may provide the abundance for from 2 to 100 microbial taxa, from 3 to 50 microbial taxa, from 4 to 25 microbial taxa, or from 5 to 10 microbial taxa, or from 6 to 8 microbial taxa.

Preferably, the subject's gut microbiota data provides the abundance for 4 or more microbial taxa, 5 or more microbial taxa, or 6 or more microbial taxa. Preferably, the subject's gut microbiota data provides the abundance for 10 or fewer microbial taxa, 9 or fewer microbial taxa, or 8 or fewer microbial taxa. Preferably, the subject's gut microbiota data provides the abundance for from 4 to 10 microbial taxa, from 5 to 9 microbial taxa, or from 6 to 8 microbial taxa. In some embodiments, the subject's gut microbiota data provides the abundance for 6 to 8 microbial taxa.

The gut microbiota data may provide the relative abundance and/or absolute abundance for the plurality of microbial taxa. Suitably, the gut microbiota data provides the relative abundance for the plurality of microbial taxa.

The subject's gut microbiota data may provide the abundance for the same microbial taxa as the gut microbiota data from the population of healthy subjects or for different microbial taxa. The microbial taxa may be classified according to any suitable classification. The microbial taxa may be classified by the same classification system(s) or by one or more different classification systems. For example, the microbial taxa may be taxonomically-classified and/or functionally-classified, as described above.

The subject's gut microbiota data may provide 2 or more (e.g. 2, 3, 4, 5, 6, 7, 8, 9, 10) microbial ratios, 3 or more microbial ratios, 4 or more microbial ratios, 5 or more microbial ratios, 6 or more microbial ratios, 7 or more microbial ratios, 8 or more microbial ratios, 9 or more microbial ratios, 10 or more microbial ratios. Suitably, the subject's gut microbiota data may provide 100 or fewer microbial ratios, 50 or fewer microbial ratios, 25 or fewer microbial ratios, 10 or fewer microbial ratios, 9 or fewer microbial ratios, 8 or fewer microbial ratios, or 7 or fewer microbial ratios. Suitably, the subjects gut microbiota data may provide from 2 to 100 microbial ratios, from 2 to 50 microbial ratios, from 3 to 25 microbial ratios, from 4 to 10 microbial ratios, or from 5 to 7 microbial ratios.

Preferably, the subject's gut microbiota data provides 3 or more microbial ratios, 4 or more microbial ratios, or 5 or more microbial ratios. Preferably, the subject's gut microbiota data provides 9 or fewer microbial ratios, 8 or fewer microbial ratios, or 7 or fewer microbial ratios. Preferably, the subjects gut microbiota data provide from 3 to 9 microbial ratios, from 4 to 8 microbial ratios, or from 5 to 7 microbial ratios.

### Outlier in a Trained Regression Model

In one aspect, the present invention provides a method for determining the gut microbiota status of a subject, wherein the method comprises determining whether the subject is an outlier or not in a trained regression model. The method may use any trained regression model described herein.

Suitably, the gut microbiota status of the subject is healthy if the subject is not an outlier in the trained regression model, and/or the gut microbiota status of the subject is not healthy if the subject is an outlier in the trained regression model.

In some embodiments, the gut microbiota status of the subject is healthy if the subject is not an outlier in the trained regression model. In this context, a “healthy” gut microbiota status means that the subject has a gut microbiota that does not differ significantly from the gut microbiota of a population of healthy subjects.

A “healthy” gut microbiota status may mean that that the subject is in an appropriate gut maturation state, is in an appropriate gut progression state, and/or is in an appropriate gut succession stage.

In one embodiment, a healthy gut microbiota status means that the subject is in an appropriate gut maturation state. An “appropriate gut maturation state” may mean that the subject's gut microbiota is maturing normally or properly.

In one embodiment, a healthy gut microbiota status means that the subject is in an appropriate gut progression state. An “appropriate gut progression state” may mean that the subject's gut microbiota is progressing or evolving in a timely manner.

In one embodiment, a healthy gut microbiota status means that the subject is in an appropriate gut succession state. An “appropriate gut succession state” may mean that the subject's gut microbiota is succeeding in a timely manner.

In some embodiments, the gut microbiota status of the subject is not healthy if the subject is an outlier. In this context, a gut microbiota status which is “not healthy” means that the subject has a gut microbiota that differs significantly from the gut microbiota of a population of healthy subjects.

A gut microbiota status which is “not healthy” may mean that that the subject is not in an appropriate gut maturation state, is not in an appropriate gut progression state, and/or is not in an appropriate gut succession stage. In one embodiment, a gut microbiota status which is not healthy means that the subject is not in an appropriate gut maturation state. In one embodiment, a gut microbiota status which is not healthy means that the subject is not in an appropriate gut progression state. In one embodiment, a gut microbiota status which is not healthy means that the subject is not in an appropriate gut succession state.

Any suitable statistical method may be used to determine whether the subject is an outlier in the trained regression model (see e.g. Hodge, V. and Austin, J., 2004. Artificial intelligence review, 22(2), pp.85-126). For example, the subject may be determined to be an outlier based on the standard errors, confidence intervals, prediction intervals, and/or standard deviations in the trained regression model. Suitably, the subject may be determined to be an outlier if their gut microbiota data differs significantly from the trained regression line, based on the standard errors, confidence intervals, prediction intervals, and/or standard deviations of the trained regression line.

Suitable cut-offs will be well known to the skilled person. For example, three standard deviations from the mean is a common cut-off in practice for identifying outliers in a Gaussian or Gaussian-like distribution.

In some embodiments, the subject is determined to be an outlier based on the standard error of the trained regression model. The standard error of the regression (SE) represents the average distance that the observed values fall from the regression line. Suitably, the subject is an outlier if their gut microbiota data is −2SE or less or 2SE or more, −3SE or less or 3SE or more, or −4SE or less or 4SE or more from the trained regression line. Suitably, the subject is an outlier if their gut microbiota data is −2SE or less or 2SE or more from the trained regression line.

In some embodiments, the subject is determined to be an outlier based on the confidence interval of the trained regression model. The confidence interval may be determined by any suitable method, for example using resampling approaches (e.g. bootstrap resampling). Suitably, the subject is an outlier if their gut microbiota data falls outside the 90% confidence interval, the 95% confidence interval, the 98% confidence interval, or the 99% confidence interval in the trained regression model. Suitably, the subject is an outlier if their gut microbiota data falls outside the 95% confidence interval in the trained regression model.

In some embodiments, the subject is determined to be an outlier based on prediction interval of the trained regression model. Suitably, the subject is an outlier if their gut microbiota data falls outside the 90% prediction interval, the 95% prediction interval, the 98% prediction interval, or the 99% prediction interval in the trained regression model. Suitably, the subject is an outlier if their gut microbiota data falls outside the 95% prediction interval in the trained regression model.

In some embodiments, the subject is determined to be an outlier based on standard deviation of the trained regression model. For example, a Z-score can be used to determine whether the subject is an outlier. The Z-score is the number of standard deviations above and below the mean. Suitably, the subject is an outlier if they have a Z-score of −2 or less or 2 or more, a Z-score of −3 or less or 3 or more, or a Z-score of −4 or less or 4 or more in the trained regression model. Suitably, the subject is an outlier if they have a Z-score of −2 or less or 2 or more in the trained regression model.

In some embodiments, the subject is determined to be an outlier if their gut microbiota data is −2SE or less or 2SE or more from the trained regression line, if their gut microbiota data falls outside the 95% confidence interval in the trained regression model, if their gut microbiota data falls outside the 95% prediction interval in the trained regression model, and/or if they have a Z-score of −2 or less or 2 or more in the trained regression model.

In some embodiments, the subject is determined to be an outlier if their gut microbiota data falls outside the 95% confidence interval in the trained regression model and/or if their gut microbiota data falls outside the 95% prediction interval in the trained regression model.

In some embodiments, the subject is determined to be an outlier if their gut microbiota data falls outside the 95% prediction interval in the trained regression model.

### On or Off an ELM Trajectory

In one aspect, the present invention provides a method for determining the gut microbiota status of a subject, wherein the method comprises determining whether the subject is on or off an ELM trajectory. The method may use any trained regression model described herein.

Suitably, the gut microbiota status of the subject is healthy if the subject is on the ELM trajectory, and/or wherein the gut microbiota status of the subject is not healthy if the subject is off the ELM trajectory.

In some embodiments, the gut microbiota status of the subject is healthy if the subject is on the ELM trajectory. In some embodiments, the gut microbiota status of the subject is not healthy if the subject is off the ELM trajectory.

Suitably, the subject is on the ELM trajectory if the subject's gut microbiota data does not differ significantly from the ELM trajectory and/or wherein the subject is off the ELM trajectory if the subject's gut microbiota data differs significantly from the ELM trajectory.

Any suitable method may be used to determine whether the subject is on the ELM trajectory. For example, the subject may be determined to be on the ELM trajectory based on the standard errors, confidence intervals, prediction intervals, and/or standard deviations of the ELM trajectory.

In some embodiments, the subject is determined to be off the ELM trajectory based on the standard error (SE) of the ELM trajectory. Suitably, the subject is off the ELM trajectory if their gut microbiota data is −2SE or less or 2SE or more, −3SE or less or 3SE or more, or −4SE or less or 4SE or more from the ELM trajectory. Suitably, the subject is off the ELM trajectory if their gut microbiota data is −2SE or less or 2SE or more from the ELM trajectory.

In some embodiments, the subject is determined to be off the ELM trajectory based on the confidence interval of the ELM trajectory. Suitably, the subject is off the ELM trajectory if their gut microbiota data falls outside the 90% confidence interval, the 95% confidence interval, the 98% confidence interval, or the 99% confidence interval of the ELM trajectory. Suitably, the subject is off the ELM trajectory if their gut microbiota data falls outside the 95% confidence interval of the ELM trajectory.

In some embodiments, the subject is determined to be off the ELM trajectory based on prediction interval of the ELM trajectory. Suitably, the subject is off the ELM trajectory if their gut microbiota data falls outside the 90% prediction interval, the 95% prediction interval, the 98% prediction interval, or the 99% prediction interval of the ELM trajectory. Suitably, the subject is off the ELM trajectory if their gut microbiota data falls outside the 95% prediction interval of the ELM trajectory

In some embodiments, the subject is determined to be off the ELM trajectory based on standard deviation of the ELM trajectory. For example, a Z-score can be used to determine whether the subject is off the ELM trajectory. Suitably, the subject is an outlier if they have a Z-score of −2 or less or 2 or more, a Z-score of −3 or less or 3 or more, or a Z-score of −4 or less or 4 or more. Suitably, the subject is off the ELM trajectory if they have a Z-score of −2 or less or 2 or more.

In some embodiments, the subject is determined to be off the ELM trajectory if their gut microbiota data is −2SE or less or 2SE or more from the ELM trajectory, if their gut microbiota data falls outside the 95% confidence interval of the ELM trajectory, if their gut microbiota data falls outside the 95% prediction interval of the ELM trajectory, and/or if they have a Z-score of −2 or less or 2 or more.

In some embodiments, the subject is determined to be off the ELM trajectory if their gut microbiota data falls outside the 95% confidence interval of the ELM trajectory model and/or if their gut microbiota data falls outside the 95% prediction interval of the ELM trajectory.

In some embodiments, the subject is determined to be off the ELM trajectory if their gut microbiota data falls outside the 95% prediction interval of the ELM trajectory.

### Predicting Subject's Age

The present invention provides a method for predicting the age of a subject given their gut microbiota data. The prediction may be based on the assumption that the subject is healthy.

The method may comprise predicting the age of the subject given their gut microbiota data and a trained regression model. The trained regression model may be any trained regression model described herein and/or maybe obtained or obtainable by any method described herein.

The method may comprise: (a) providing gut microbiota data from a population of healthy subjects; (b) training a regression model on the gut microbiota data, wherein the age of the healthy subjects at data collection is regressed on one or more microbial ratios provided from the gut microbiota data; (c) providing gut microbiota data from a subject of interest; and (d) predicting the age of the subject of interest given their gut microbiota data and the trained regression model.

The present invention provides a method for determining the gut microbiota status of a subject, wherein the gut microbiota status of the subject is healthy if the predicted age of the subject does not differ significantly from the actual age of the subject and/or wherein the gut microbiota status of the subject is not healthy if the predicted age of the subject differs significantly from the actual age of the subject.

Any suitable method may be used to determine whether the predicted age of the subject differs significantly from the actual age of the subject, for example based on standard errors, confidence intervals, prediction intervals, and/or standard deviations of the trained regression model (as described above in more detail).

Suitably, the predicted age of the subject differs significantly from the actual age of the subject if their predicted age is −2SE or less or 2SE or more, −3SE or less or 3SE or more, or −4SE or less or 4SE or more from their actual age. Suitably, the predicted age of the subject differs significantly from the actual age of the subject if their predicted age is −2SE or less or 2SE or more from their actual age.

Suitably, the predicted age of the subject differs significantly from the actual age of the subject has an age Z-score of −2 or less or 2 or more, an age Z-score of −3 or less or 3 or more, or an age Z-score of −4 or less or 4 or more. Suitably, the predicted age of the subject differs significantly from the actual age of the subject if the subject has an age Z-score of −2 or less or 2 or more.

Suitably, the predicted age of the subject differs significantly from the actual age of the subject if it differs by about 1 month or more (e.g. 1, 2, 3, 4, 5, 6, 7, 8, 9, 10, 11, or 12 months), by about 2 months or more, by about 3 months or more, by about 4 months or more, by about 5 months or more, 6 months or more, by about 7 months or more, by about 8 months or more, by about 9 months or more, by about 10 months or more, by about 11 months or more, or by about 12 months or more. Suitably, the predicted age of the subject differs significantly from the actual age of the subject if it differs by about 0.5 years or more (e.g. 0.5, 0.6, 0.7, 0.8, 0.9, or 1 year), by about 0.6 years or more, by about 0.7 years or more, by about 0.8 years or more, by about 0.9 years or more, or by about 1 year or more.

The present invention also provides use of one or more microbial ratios provided from a subject's gut microbiota data to predict the age of the subject. The present invention also provides use of a trained regression model according to the present invention to predict the age of a subject.

### Method for Maintaining or Improving the Gut Microbiota Status of a Subject

The present invention provides a method for maintaining or improving the gut microbiota status of a subject.

The method may comprise determining the gut microbiota status of the subject using any method described herein and adjusting the diet, nutrient intake, and/or lifestyle of the subject to maintain or improve the subject's gut microbiota status.

After adjusting the diet, nutrient intake, and/or lifestyle of the subject the gut microbiota status of the subject may be healthy. After adjusting the diet, nutrient intake, and/or lifestyle of the subject the subject may be in an appropriate gut maturation state, in an appropriate gut progression state, and/or in an appropriate gut succession stage.

The adjusted diet, nutrient intake, and/or lifestyle of the subject may change one or more microbial ratios. Suitably, the microbial ratios are ones which have a high impact on the trained regression model and/or ones which differ significantly from the median microbial ratios in the trained regression model. In some embodiments, the adjusted diet, nutrient intake, and/or lifestyle of the subject may change microbial ratios which have a high impact on the trained regression model and which differ significantly from the median microbial ratios in the trained regression model.

Any suitable statistical method may be used to identify microbial ratios which have a high impact on the trained regression model, for example based on the feature importance. The feature importance may be determined using any suitable statistical method, for example based on SHapley Additive explanation (SHAP) values (Lundberg S M, et al. Nat Mach Intell. 2020).

Any suitable statistical method may be used to identify microbial ratios which differ significantly from the median microbial ratios in the trained regression model, for example based on standard errors, confidence intervals, prediction intervals, and/or standard deviations (as described above in more detail).

The adjusted diet, nutrient intake, and/or lifestyle of the subject may increase the abundance and/or function of one or more favourable microbial taxa and/or to decrease the abundance and/or function of one or more unfavourable microbial taxa.

Suitably, the adjusted diet, nutrient intake, and/or lifestyle of the subject may increase the abundance and/or function of one or more favourable microbial taxa. In this context, “favourable microbial taxa” may be microbial taxa which have a lower microbial ratio in the subject's gut microbiota data than in a population of healthy subjects. For example, favourable microbial taxa may be the microbial taxa that have a lower microbial ratio in the subject's gut microbiota data compared to the median microbial ratio in a trained regression model. Suitably, the favourable microbial taxa may include Bifidobacterium (e.g. when the subject is 0-12 months of age or 0-6 months of age). Bifidobacterium is an important component of an infant's gut microbiota in the first few months of life. Certain species of Bifidobacterium benefit from Human Milk Oligosaccharides (HMOs) (Berger, B., et al., 2020. Mbio, 11(2)). Suitably, the favourable microbial taxa may include Faecalibacterium, e.g. Faecalibacterium prausnitzii (e.g. when the subject is at least 12 months of age). Faecalibacterium prausnitzii starts establishing in the infant's gut from about 6 months to becomes a predominant member of the gut microbiome from about 2 years onwards (Miquel, S., et al., 2014. Gut microbes, 5(2), pp. 146-151; Laursen et al. mSphere 2017). Faecalibacterium beyond 12 months of age was related to higher diversity in the infant's gut ecosystem (Roswall, J., et al., Cell Host & Microbe, 2021.).

Suitably, the adjusted diet, nutrient intake, and/or lifestyle of the subject may decrease the abundance and/or function of one or more unfavourable microbial taxa. In this context, “unfavourable microbial taxa” may be microbial taxa which have a higher microbial ratio in the subject's gut microbiota data than in a population of healthy subjects. For example, unfavourable microbial taxa may be the microbial taxa that have a higher microbial ratio in the subject's gut microbiota data compared to the median microbial ratio in a trained regression model. Suitably, the unfavourable microbial taxa may be Bacteroides.

Suitably, after adjusting the diet, nutrient intake, and/or lifestyle of the subject the gut microbiota status of the subject is healthy. The gut microbiota status may be determined using any method described herein (e.g. the same method used to determine the gut microbiota status prior to adjusting the diet, nutrient intake, and/or lifestyle of the subject).

The present invention also provides a method for determining a subject's diet and/or nutrient intake. The method may comprise determining the diet and/or nutrient intake required to maintain or improve the gut microbiota status of the subject.

These personalised recommendations may change over time as the subject's gut microbiota over time. The methods of the present invention may be used one or more times (e.g. 1, 2, 3, 4, 5, 6, 7, 8, 9, 10) or two or more times when the subject of interest is from 0 years to 1 year of age, 0.5 years to 1 year of age, or 1 year to 2 years of age. The methods of the present invention may be used one or more times (e.g. 1, 2, 3, 4, 5, 6, 7, 8, 9, 10) or two or more times when the subject of interest is from 0 months to 12 months of age, 6 months to 12 months of age, or 12 months to 24 months of age. The methods of the present invention may be used two times or more (e.g. 2, 3, 4, 5, 6, 7, 8, 9, 10 times), three times or more, four times or more, or five times or more, in the first 5 years of a subject's life. For example, the methods of the present invention may be used two times or more (e.g. 2, 3, 4, 5, 6, 7, 8, 9, 10 times), three times or more, four times or more, or five times or more, in the first 2 years of life.

### Diet

In one aspect, the present invention provides a method for maintaining or improving the gut microbiota status comprising adjusting the diet of the subject. For example, the subject may be provided a diet recommendation.

In one aspect, the present invention provides a method for determining a subject's diet. The method may comprise determining the diet required to maintain or improve the gut microbiota status of the subject.

Methods of the invention may comprise determining a diet to increase the abundance and/or function of one or more favourable microbial taxa and/or to decrease the abundance and/or function of one or more unfavourable microbial taxa.

Methods of the invention may comprise administering a diet to increase the abundance and/or function of one or more favourable microbial taxa and/or to decrease the abundance and/or function of one or more unfavourable microbial taxa.

The subject's “diet” may include all the food consumed by the subject. It is known that diet has a major impact on gut microbiota composition, diversity, and richness.

The subject's diet may provide a plurality of food groups. The term “food group” may refer to a collection of foods that share similar nutritional properties or biological classifications. Nutrition guides typically divide foods into food groups and Recommended Dietary Allowance recommend daily servings of each group for a healthy diet. Exemplary food groups include fruits; vegetables; pulses, nuts or seeds; meats; starches or grains; dairy; and oils and fats.

The subject's diet may also provide a plurality of food types. The term “food type” may refer to a collection of foods from the same food group that share more similar nutritional properties or biological classifications. Each food group may be further grouped into a plurality of food types. Exemplary food types for the food group fruit can include apples, banana, citrus, berries, other fruits (e.g. pear, peach, pineapple), and dried fruits. Suitable food groups and food types can be readily determined by any suitable method known in the art. For example, suitable food groups and food types can be based on published observations (e.g. Dwyer J T. The Journal of Nutrition. 2018; 148(suppl 3): 1575S-80S).

Suitably, the subject's diet may be adjusted by changing the amount of one or more food group and/or one or more food type in the subject's diet. Correlations are known between diet and microbial taxa (see e.g. Wu, G. D., et al., 2011. Science, 334(6052), pp. 105-108). For example, diets with increased fibre may stimulate growth of Bifidobacterium and Lactobacillus (see e.g. Wegh, C. A., et al., 2017. Expert review of gastroenterology & hepatology, 11(11), pp. 1031-1045).

Suitably, the subject is recommended and/or administered food to adjust the diet. In some embodiments, the food comprises dietary fibre (e.g. carbohydrate polymers, oligomers, and lignin that escape digestion in the small intestine and reach the colon intact). In some embodiments, the food comprises vitamins, such as Riboflavin (Vitamin B2), Retinol (Vitamin A), and Calciferol (Vitamin D), and/or minerals, such as Manganese, Zinc, and Potassium.

### Nutrient Intake and Supplements

In one aspect, the present invention provides a method for maintaining or improving the gut microbiota status comprising adjusting the nutrient intake of the subject. For example, the subject may be provided a nutrient or supplement recommendation (e.g. meal plans or recipes).

In one aspect, the present invention provides a method for determining a subject's nutrient intake. The method may comprise determining the nutrient intake required to maintain or improve the gut microbiota status of the subject.

Methods of the invention may comprise determining a nutrient intake to increase the abundance and/or function of one or more favourable microbial taxa and/or to decrease the abundance and/or function of one or more unfavourable microbial taxa.

Methods of the invention may comprise administering a nutrient or supplement to increase the abundance and/or function of one or more favourable microbial taxa and/or to decrease the abundance and/or function of one or more unfavourable microbial taxa.

The term “nutrient” may refer to any substance which is essential for growth and health of a subject. The term nutrient encompasses “macronutrients”, such as carbohydrates, fats and fatty acids, and proteins and “micronutrients”, such as vitamins and minerals. The subject's “nutrient intake” may include all the nutrients consumed by the subject.

Exemplary macronutrients include carbohydrates (including fibre and sugars), protein, and lipids (including long chain polyunsaturated fatty acids).

Exemplary micronutrients include vitamins (including vitamin A, vitamin D, vitamin C, folate, vitamin B6, vitamin B12, and vitamin E) and minerals (including sodium, potassium, calcium, iron, zinc, magnesium, and phosphorus).

As used herein, a “supplement” or “dietary supplement” may be used to complement the nutrition of a subject (it is typically used as such but it might also be added to any kind of compositions intended to be ingested by the subject). The supplement may be in any form suitable for intake by the subject and may comprise any suitable nutrients.

Suitably, the subject's nutrient intake may be adjusted by changing the amount of one or more nutrient in the subject's diet and/or by providing a dietary supplement. Correlations are known between nutrients and microbial taxa (see e.g. Wu, G. D., et al., 2011. Science, 334(6052), pp. 105-108). For example, it is known that human milk oligosaccharides can negatively and positively regulate gut microbiota (see e.g. Sela, D. A. and Mills, D. A., 2010. Trends in microbiology, 18(7), pp.298-307). Further, different fibre ingredients (e.g. FOS, GOS, inulin, oligofructose) have been reported to have beneficial effects on Bifidobacterium and Faecalibacterium in human studies (see e.g Lordan, C., et al., 2020. Gut Microbes, 11(1), pp. 1-20; and Verhoog, S., et al., 2019. Nutrients, 11(7), p.1565). For example, an inulin/oligofructose mix (50-50), 16g for 3 months, has been shown to increase Bifidobacterium and Faecalibacterium and decrease Bacteroides. In another intervention example, in 1 year old at 2g/day, kestose (smallest fructooligosaccharide (FOS) glucose-fructose-fructose) increased Faecalibacterium prausnitzii (Koga, Y., et al., 2016. Pediatric research, 80(6), pp.844-851). Other examples of nutrients which can regulate gut microbiota are provided in Table 2.

Suitably, the subject is administered food and/or supplements to adjust the nutrient intake. In some embodiments, the food and/or supplements comprise prebiotics (e.g. human milk oligosaccharides and/or inulin), probiotics, synbiotics, vitamins (e.g. Riboflavin (Vitamin B2), Retinol (Vitamin A), and/or Calciferol (Vitamin D)) and/or minerals (e.g. Manganese, Zinc, and/or Potassium).

### Prebiotics, Probiotics, and Synbiotics

In some embodiments, the food and/or supplements comprise prebiotics, probiotics, and/or synbiotics. Any suitable prebiotic, probiotic, and/or synbiotic may be used (see e.g. Thomas, D. W. and Greer, F. R., 2010. Pediatrics, 126(6), pp. 1217-1231).

The term “prebiotic” may refer to a non-digestible component that benefits the subject by selectively stimulating the favourable growth and/or activity of one or more microbial taxa. Exemplary prebiotics include human milk oligosaccharides. Exemplary prebiotic oligosaccharides include galacto-oligosaccharides (GOS), fructo-oligosaccharides (FOS), 2′-fucosyllactose, lacto-N-neo-tetraose, and inulin.

The term “probiotic” may refer to a component that contains a sufficient number of viable microorganisms to alter the gut microbiota of the subject (see e.g. Hill, C., et al., 2014. Nature reviews Gastroenterology & hepatology, 11(8), p.506). Exemplary probiotic microoganisms may include Escherichia, Bacteroides, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Citrobacter, Neisseria, Bifidobacterium, Lachnospira, Dialister, Ruminococcus, Blautia, Streptococcus, Eggerthella, Enterococcus and Paraprevotella.

In some embodiments, the probiotic comprises a commercially available probiotic strain and/or a strain which has been shown to have health benefits (See e.g. Fijan, S., 2014. International journal of environmental research and public health, 11(5), pp.4745-4767). In some embodiments, the probiotic comprises Escherichia, Bifidobacterium, Streptococcus, and/or Enterococcus. In some embodiments, the probiotic comprises one or more strain selected from: E. coli Nissle 1917, B. infantis, B. animalis subsp. lactis, B. bifidum, B. longum, B. breve, S. thermophilus, E. durans, and, E. faecium.

The term “synbiotic” may refer to a component that contains both probiotics and prebiotics (see e.g. Swanson, K. S., et al., 2020. Nature Reviews Gastroenterology & Hepatology, 17(11), pp.687-701).

### Vitamins and Minerals

In some embodiments, the food and/or supplements comprise vitamins and/or minerals. Dietary guidelines have been established for certain vitamins and minerals.

Exemplary vitamins include vitamin A, vitamin D, vitamin C, folate, vitamin B2, vitamin B6, vitamin B12, and vitamin E. For example, the vitamins may comprise or consist of Riboflavin (Vitamin B2), Retinol (Vitamin A), and/or Calciferol (Vitamin D).

Exemplary minerals include sodium, potassium, calcium, iron, zinc, magnesium, and phosphorus. For example, the minerals may comprise or consist of manganese, zinc, and/or potassium. Minerals are usually used in their salt form

### Lifestyle

In one aspect, the present invention provides a method for maintaining or improving the gut microbiota status comprising adjusting the lifestyle of the subject. For example, the subject may be provided a lifestyle recommendation.

Methods of the invention may comprise adjusting the subject's lifestyle to increase the abundance and/or function of one or more favourable microbial taxa and/or to decrease the abundance and/or function of one or more unfavourable microbial taxa.

By the term “lifestyle” is meant any lifestyle choice made by a subject (or subject's caretakers), and may include dietary intake data, data from questionnaires of lifestyle, motivation or preferences. For example, a lifestyle characteristic may be whether the subject is a vegan or an omnivore, whether the subject is lactose intolerant or not, frequency of physical activity, and/or frequency of sedentary activity.

Suitably, the subject's lifestyle may be adjusted by changing the subject's meal frequency and timing and/or frequency of physical activity. For example, it is known that a regular meal pattern may modulate gut microbiota (see e.g. Paoli, A., et al., 2019. Nutrients, 11(4), p.719) and that exercise may exert an influence on gut microbiota (see e.g. O'Sullivan, O., et al., 2015. Gut microbes, 6(2), pp. 131-136).

### Computer Program and Computer-Readable Medium

The methods described may be computer-implemented methods.

In one aspect, the present invention provides a data processing system comprising means for carrying out a method of the invention.

In one aspect, the present invention provides a data processing apparatus comprising a processor configured to perform a method of the invention.

In one aspect, the present invention provides a computer program comprising instructions which, when the program is executed by a computer, cause the computer to carry out a method of the invention.

In one aspect, the present invention provides a computer-readable medium comprising instructions which, when executed by a computer, cause the computer to carry out a method of the invention.

In one aspect, the present invention provides a computer-readable data carrier having stored thereon the computer program of the invention.

In one aspect, the present invention provides a data carrier signal carrying the computer program of the invention.

The systems described herein may display a dashboard or other appropriate user interface to a user that is customized based on the subject of interest. For example, based on the subject's gut microbiota samples, the subject's determined gut microbiota status, and the subject's personalized advise and recommendations such as nutritional solutions to maintain or improve the subject's gut microbiota status.

### Providing a Trained Regression Model

The methods described herein for determining a trained regression model may be computer-implemented methods.

For example, in one aspect, the present invention provides a computer-implemented method for providing a trained regression model, wherein the method comprises: (a) providing gut microbiota data from a population of healthy subjects; and (b) training a regression model on the gut microbiota data, wherein the age of the healthy subjects at data collection is regressed on one or more microbial ratios provided from the gut microbiota data.

In one aspect, the present invention provides a data processing system comprising means for determining a trained regression model given gut microbiota data from a population of healthy subjects providing the age of the healthy subjects at data collection and on one or more microbial ratios, as described herein.

In one aspect, the present invention provides a data processing apparatus comprising a processor configured to determine a trained regression model given gut microbiota data from a population of healthy subjects providing the age of the healthy subjects at data collection and on one or more microbial ratios, as described herein.

In one aspect, the present invention provides a computer program comprising instructions which, when the program is executed by a computer, cause the computer to determine a trained regression model given gut microbiota data from a population of healthy subjects providing the age of the healthy subjects at data collection and on one or more microbial ratios, as described herein.

In one aspect, the present invention provides a computer-readable medium comprising instructions which, when executed by a computer, cause the computer to determine a trained regression model given gut microbiota data from a population of healthy subjects providing the age of the healthy subjects at data collection and on one or more microbial ratios, as described herein.

### Predicting the Age of a Subject

The methods described herein predicting the age of a subject may be computer-implemented methods.

For example, in one aspect, the present invention provides a computer-implemented method for predicting the age of a subject, wherein the method comprises: (a) providing a trained regression model according to the present invention; (b) providing gut microbiota data from the subject; and (c) predicting the age of the subject given their gut microbiota data and the trained regression model.

In one aspect, the present invention provides a data processing system comprising means for predicting the age of a subject given a trained regression model according to the present invention and their gut microbiota data, as described herein.

In one aspect, the present invention provides a data processing apparatus comprising a processor configured to predict the age of a subject given a trained regression model according to the present invention and their gut microbiota data, as described herein.

In one aspect, the present invention provides a computer program comprising instructions which, when the program is executed by a computer, cause the computer to predict the age of a subject given a trained regression model according to the present invention and their gut microbiota data, as described herein.

In one aspect, the present invention provides a computer-readable medium comprising instructions which, when executed by a computer, cause the computer to predict the age of a subject given a trained regression model according to the present invention and their gut microbiota data, as described herein.

### Determining the Gut Microbiota Status of a Subject

The methods described herein for determining the gut microbiota status of a subject may be computer-implemented methods.

### Outlier in a Trained Regression Model

In one aspect, the present invention provides a computer-implemented method for determining the gut microbiota status of a subject, wherein the method comprises: (a) providing a trained regression according to the present invention; (b) providing gut microbiota data from the subject; and (c) determining whether the subject is an outlier or not in the trained regression model; wherein the gut microbiota status of the subject is healthy if the subject is not an outlier in the trained regression model, and/or wherein the gut microbiota status of the subject is not healthy if the subject is an outlier.

In one aspect, the present invention provides a data processing system comprising means for determining the gut microbiota status of a subject given a trained regression according to the present invention and their gut microbiota data, as described herein.

In one aspect, the present invention provides a data processing apparatus comprising a processor configured to determine the gut microbiota status of a subject given a trained regression according to the present invention and their gut microbiota data, as described herein.

In one aspect, the present invention provides a computer program comprising instructions which, when the program is executed by a computer, cause the computer to determine the gut microbiota status of a subject given a trained regression according to the present invention and their gut microbiota data, as described herein.

In one aspect, the present invention provides a computer-readable medium comprising instructions which, when executed by a computer, cause the computer to determine the gut microbiota status of a subject given a trained regression according to the present invention and their gut microbiota data, as described herein.

### On or Off an ELM Trajectory

In one aspect, the present invention provides a computer-implemented method for determining the gut microbiota status of a subject, wherein the method comprises: (a) providing a trained regression model according to the present invention, wherein the trained regression model is an ELM trajectory; (b) providing gut microbiota data from the subject; and (c) determining whether the subject is on or off the ELM trajectory; wherein the subject is on the ELM trajectory if the subject's gut microbiota data does not differ significantly from the ELM trajectory and/or wherein the subject is off the ELM trajectory if the subject's gut microbiota data differs significantly from the ELM trajectory.

In one aspect, the present invention provides a data processing system comprising means for determining the gut microbiota status of a subject given an ELM trajectory according to the present invention and their gut microbiota data, as described herein.

In one aspect, the present invention provides a data processing apparatus comprising a processor configured to determine the gut microbiota status of a subject given an ELM trajectory according to the present invention and their gut microbiota data, as described herein.

In one aspect, the present invention provides a computer program comprising instructions which, when the program is executed by a computer, cause the computer to determine the gut microbiota status of a subject given an ELM trajectory according to the present invention and their gut microbiota data, as described herein.

In one aspect, the present invention provides a computer-readable medium comprising instructions which, when executed by a computer, cause the computer to determine the gut microbiota status of a subject given an ELM trajectory according to the present invention and their gut microbiota data, as described herein.

### Predicted Age Differs Significantly

In one aspect, the present invention provides a computer-implemented method for determining the gut microbiota status of a subject, wherein the method comprises predicting the age of the subject by a method according to the present invention, and wherein the gut microbiota status of the subject is healthy if the predicted age of the subject does not differ significantly from the actual age of the subject and/or wherein the gut microbiota status of the subject is not healthy if the predicted age of the subject differs significantly from the actual age of the subject.

In one aspect, the present invention provides a data processing system comprising means for determining the gut microbiota status of a subject given their predicted according to the present invention and their actual age.

In one aspect, the present invention provides a data processing apparatus comprising a processor configured to determine the gut microbiota status of a subject given their predicted according to the present invention and their actual age.

In one aspect, the present invention provides a computer program comprising instructions which, when the program is executed by a computer, cause the computer to determine the gut microbiota status of a subject given their predicted according to the present invention and their actual age.

In one aspect, the present invention provides a computer-readable medium comprising instructions which, when executed by a computer, cause the computer to determine the gut microbiota status of a subject given their predicted according to the present invention and their actual age.

### EXAMPLES

The invention will now be further described by way of examples, which are meant to serve to assist the skilled person in carrying out the invention and are not intended in any way to limit the scope of the invention.

### Example 1: Choosing a Reference Microbial Taxon

The microbial taxon chosen to be the reference (as a factor that goes in the denominator for ratio calculations) can be based on number of crossings this microbial taxon has with all the other microbial taxa in the dataset (explained in the next paragraph) and/or on other criteria such as modelling performance statistics, availability of primers for qPCR test, and so on.

Since this chosen microbial taxon must go in the denominator for the ratio calculation, we check its abundance over time and how that compares, one by one, with the abundance over time for all the other microbial taxa in the dataset. If the abundance profiles of these taxa over time are crossing or interweaving each other quite a few times, that is not good for the calculations as the ratios will keep changing from >1 to <1 as per how the abundances changed or crossed-over each other. Thus, it will create frequent numerical changes in the ratio transformation we are doing in the dataset. With this reasoning, we prioritize picking those microbial taxa to go as denominator in the ratio calculations, which have minimum number of crossings with all other microbial taxa.

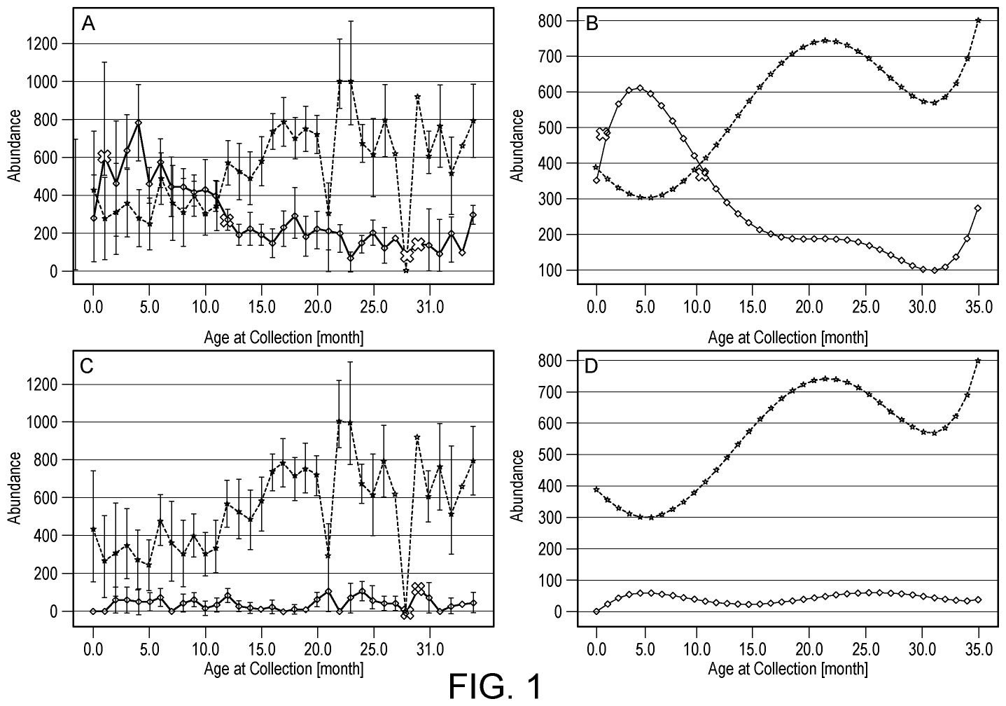

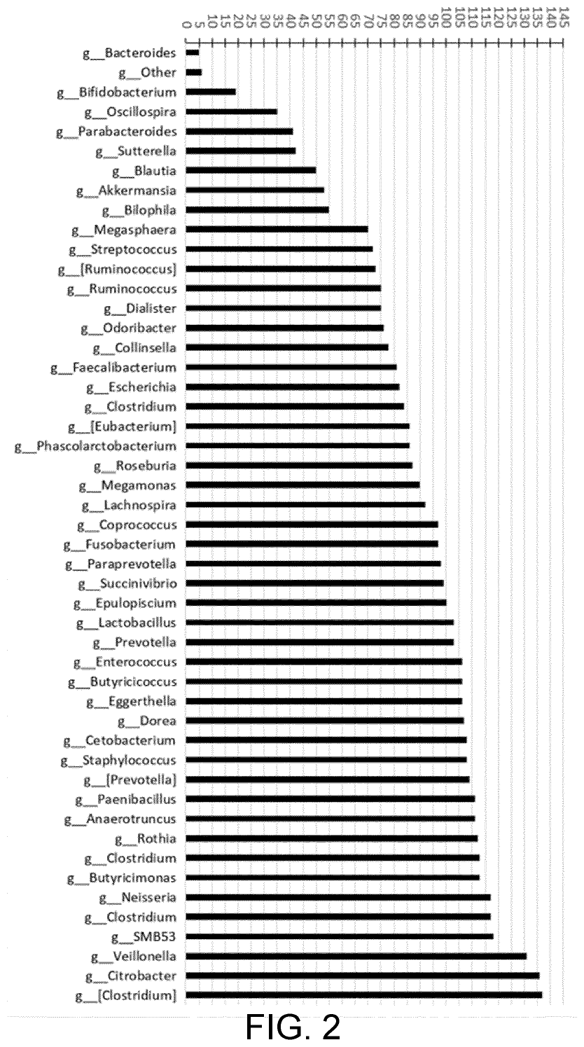

This is visually shown in FIG. 1 to indicate how the abundances of two microbial taxa cross or interweave with each other over time (or not). FIG. 2 depicts the total number of crossings found for each microbial taxon when compared against all the other microbial taxa.

### Example 2: Derivation of an Early Life Microbiome (ELM) Trajectory Using Genus Level Data and Ratios Derived Using Escherichia in the Denominator

Using 16S Genus level microbiota data from an American cohort called BCP-Enriched, which is an ancillary study to BCP-study (Howell B R, et al. Neuroimage. 2019) with expanded scope to explore nutritional impacts, we defined a healthy reference set as those infants who were Breast Fed. The UNC/UMN Baby Connectome Project (BCP) is an ongoing study jointly conducted by investigators at the University of North Carolina at Chapel Hill and the University of Minnesota and involving a total sample of 500 typically developing infants, toddlers, and preschool-aged children recruited and enrolled between birth and 5 years of age. There were 91 samples in the healthy reference set from 52 infants. A RandomForest machine learning model was trained to predict the microbiota age with the following Train data size: 40 samples from 26 infants. For fitting of the ELM trajectory, the Validation data was used: 51 samples from 26 infants. Lastly, for testing the model and trajectory, the Test data was used:—298 samples from 127 infants. The log ratio of all the microbial taxa in the microbiota data for these infants is done with genus Escherichia.

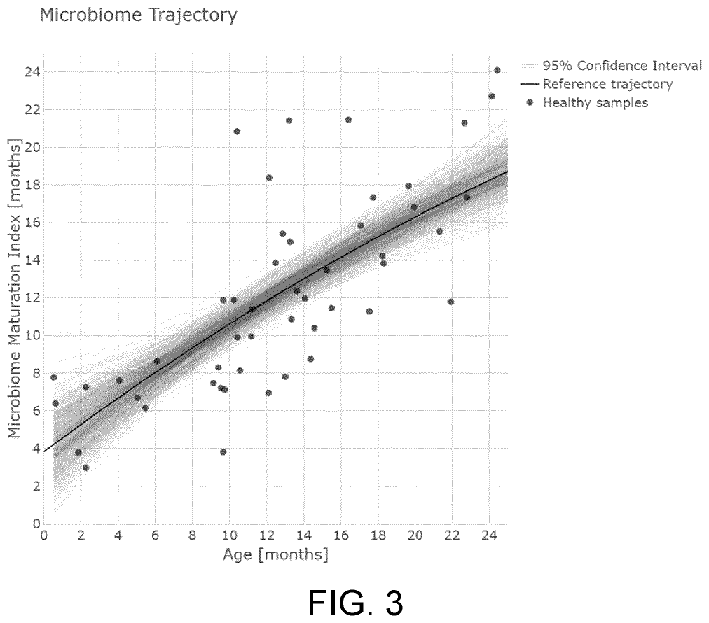

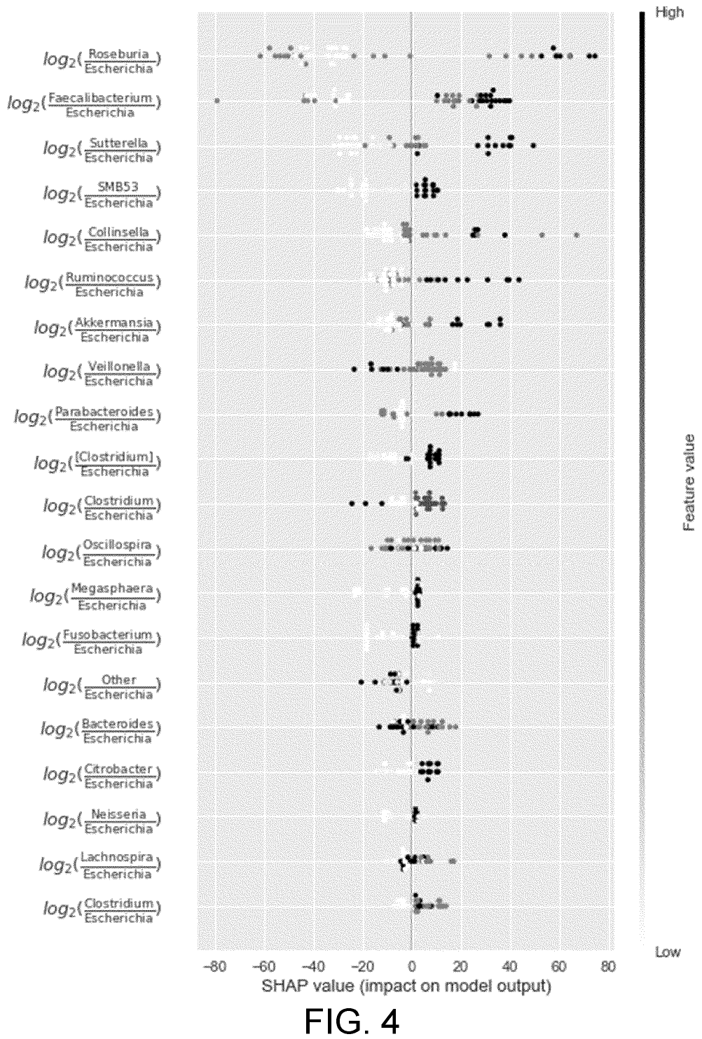

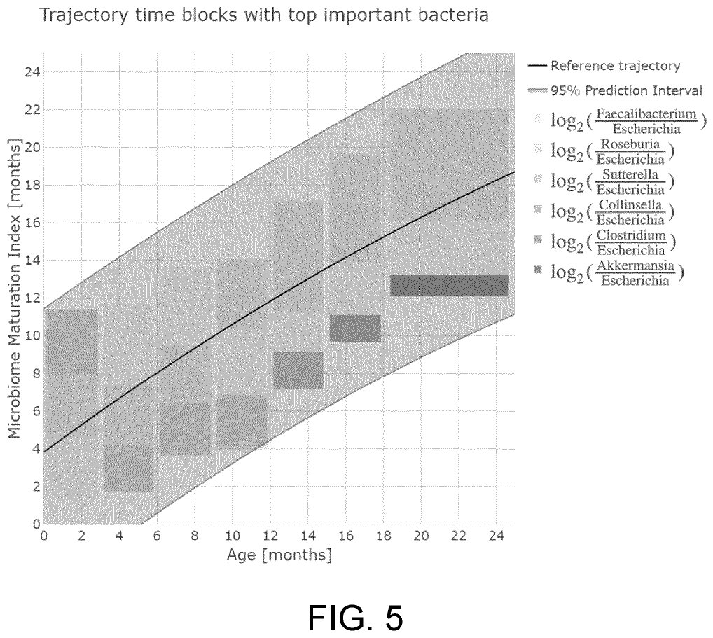

The results obtained for the ELM trajectory are shown in FIG. 3. The important features (key microbial ratios) constituting the model are shown in FIG. 4 in their order of importance from top to bottom. The key microbial ratios per different time windows are shown in FIG. 5.

### Example 3: Derivation of an Early Life Microbiome (ELM) Trajectory Using Genus Level Data and Ratios Derived Using Bacteroides in the Denominator

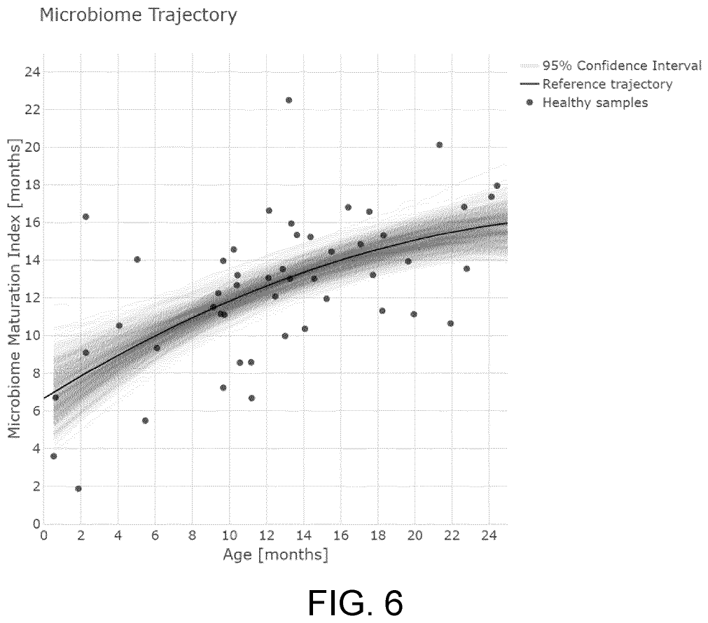

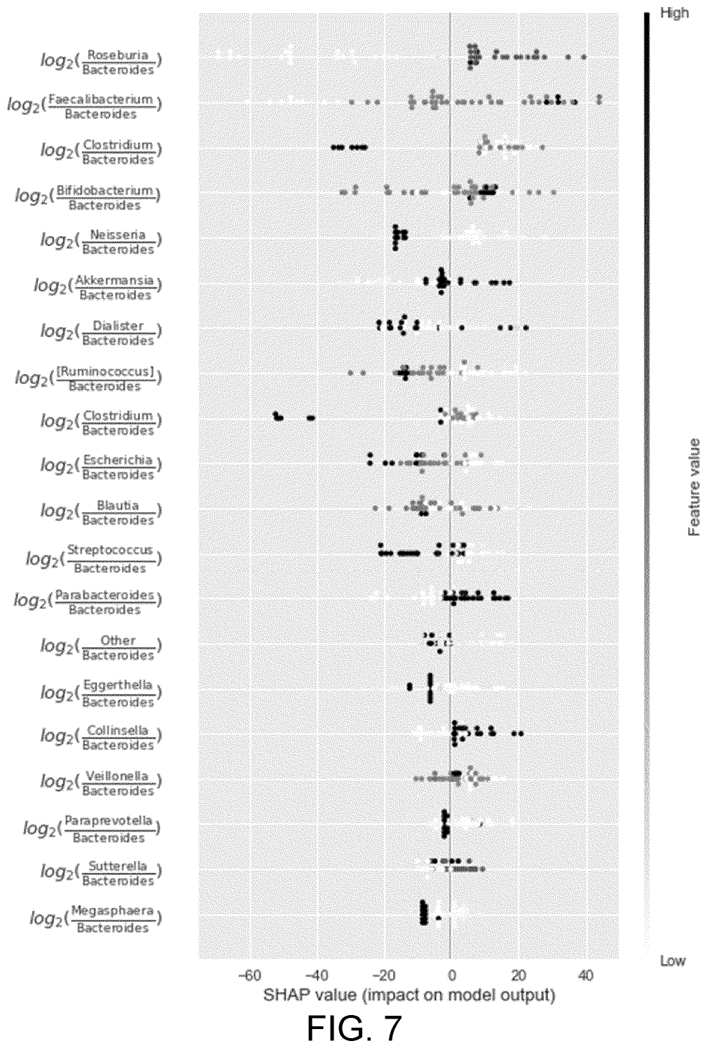

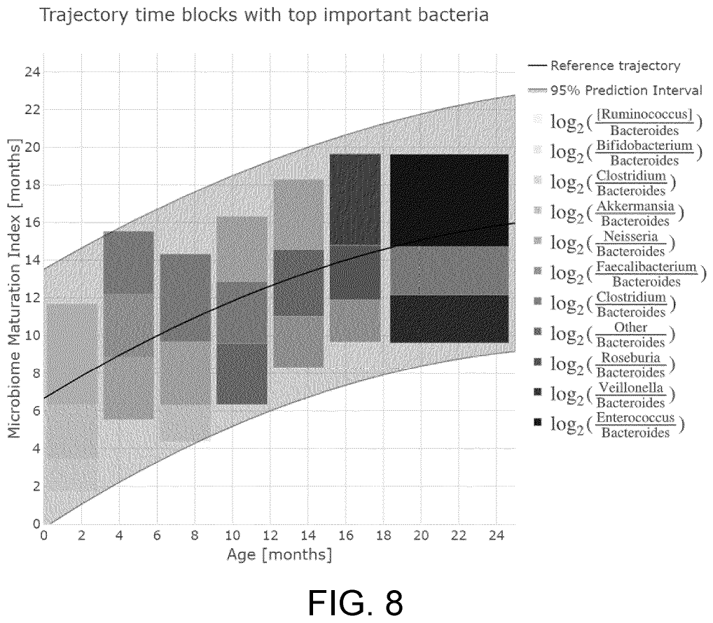

Using 16S Genus level microbiota data, as discussed in Example 2, we now instead derived the ratios using a different genus, Bacteroides. The results obtained for the ELM trajectory are shown in FIG. 6. The important features (key microbial ratios) constituting the model are shown in FIG. 7 in their order of importance from top to bottom. The key microbial ratios per different time windows are shown in FIG. 8.

### Example 4: Identification of samples off the Early Life Microbiome (ELM) Trajectory

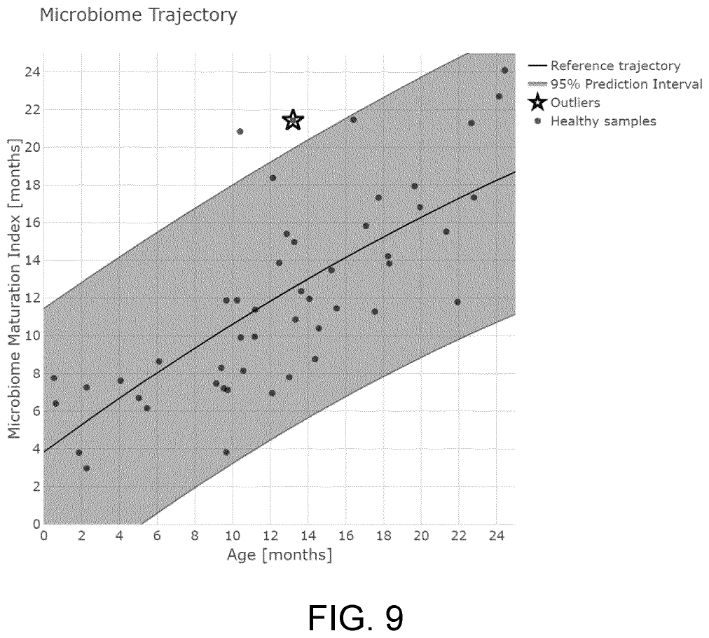

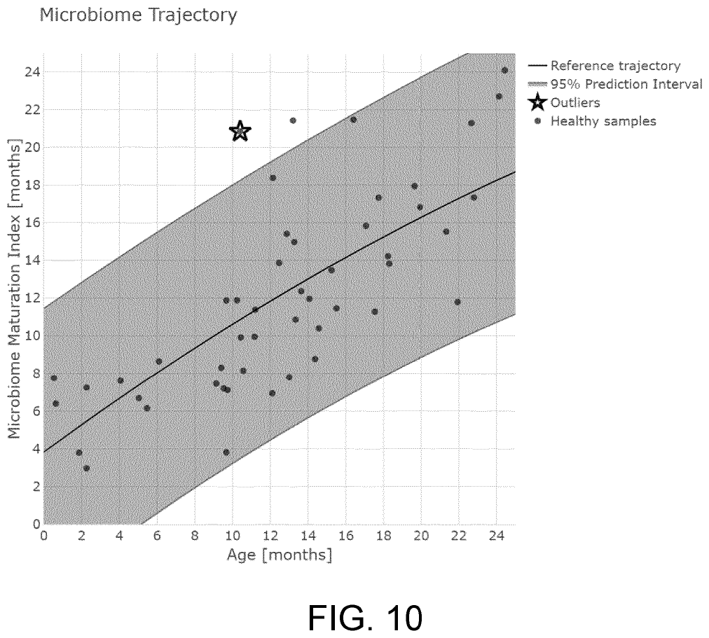

As shown in FIG. 5 related to Example 2, there are some samples (infant at given time-point), who are off the trajectory. An example of off the trajectory sample is shown in FIG. 9. Another example of an off the trajectory sample is shown in FIG. 10.

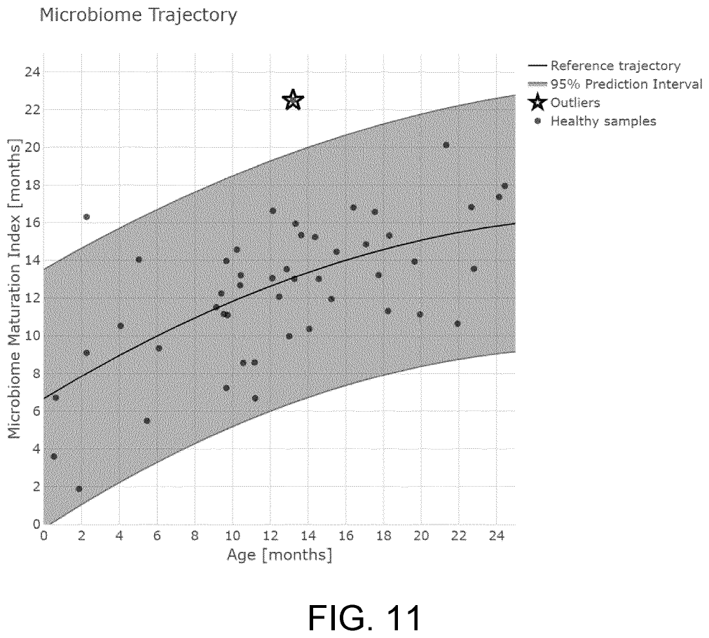

As shown in FIG. 8 related to Example 3, there are some samples (infant at given time-point), who are off the trajectory. An example of off the trajectory sample is shown in FIG. 11. Another example of an off the trajectory sample is shown in FIG. 12.

### Example 5: Intervention to Bring Back Off the Early Life Microbiome (ELM) Trajectory Samples Back on the ELM Trajectory

The off the ELM trajectory samples happen to be so because these do not have the key microbes that constitute the microbiota compositional age model in the appropriate amounts and ranges as found for the healthy reference infants in the same age group. Thus, intervening by various means, for example, nutritional supplements such as vitamins and minerals that improve the abundance and function of these microbes, should restore these microbial taxa to their normal amounts. This should in principle then lead to the sample being placed back on the ELM trajectory.

Indeed, this is shown by simulations where we first looked at the few key microbial ratios for the outlier samples, then checked which of these are out of range compared to the reference in the same age group (time window). Next, we changed these few key microbial ratios to be the same as the median of these microbial ratios for the reference infants in the same age group (time-window). Lastly, we re-did the predictions using the model to see if this now brings these outlier samples back on the ELM trajectory.

For example, for the off the trajectory example presented in FIG. 11, in Table 1, the column “Feature importance value for outlier” contains the importance values of key microbial taxa for the outlier sample. These microbial taxa have the highest effect on the sample for it to be an outlier. Column “Feature importance value” contains the importance values of microbial taxa in the trajectory region where we want to place back this outlier for it to be normal based on the time-window that the outlier belongs to. Sorting by these two columns, accenting the one for the “Feature importance value for outlier”, we get top 5 important microbial taxa to change. Sorting on the second column “Feature importance value”, helps to resolve any conflicts in choosing the top 5 microbial taxa when only sorted on the first column “Feature importance value for outlier”. To make these outliers go back in to the trajectory, we only use these top 5 microbial taxa for simulation-based intervention, where we change the “Taxa value for outlier” to be the same as “Taxa average” which is calculated from the reference samples in the same time-window as the outlier sample is.

TABLE 1
importance values of microbial taxa for the outlier sample

| | | | | Feature |
|---|---|---|---|---|
| | Feature | | | importance |
| | importance | Taxa | Taxa value | value for |
| Taxa | value | average | for outlier | outlier |
| Clostridium | 0.20 | −38.96 | −4.54 | 17.38 |
| [Ruminococcus] | 10.04 | −3.66 | −42.08 | 10.45 |
| Escherichia | 4.32 | −2.56 | −42.08 | 8.78 |
| Parabacteroides | 2.20 | −41.67 | −0.78 | 7.04 |
| Veillonella | 0.14 | −2.19 | −42.08 | 4.95 |

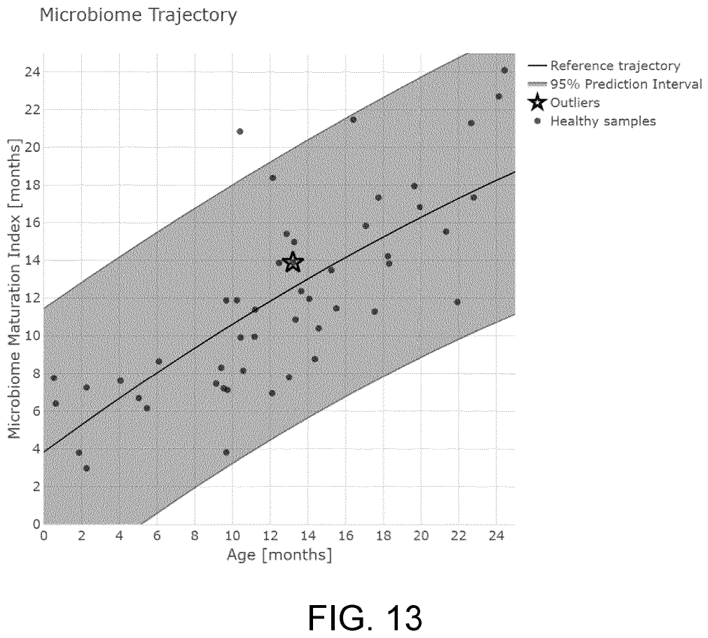

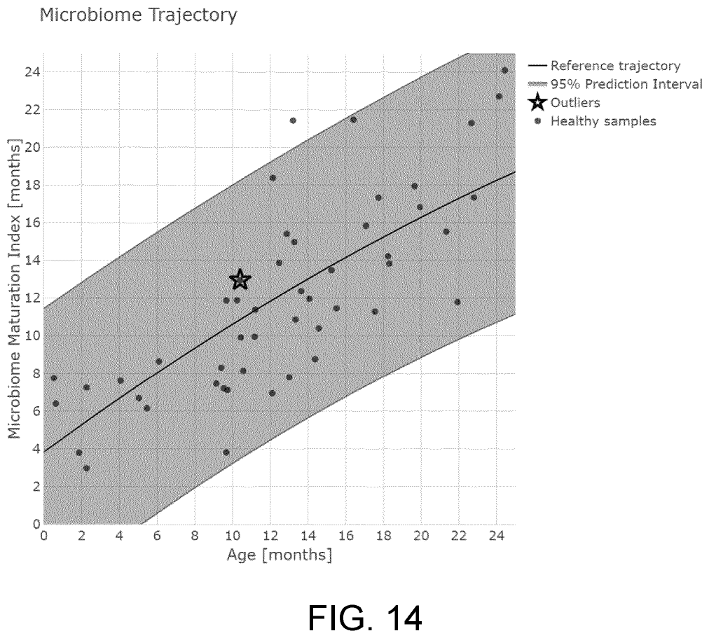

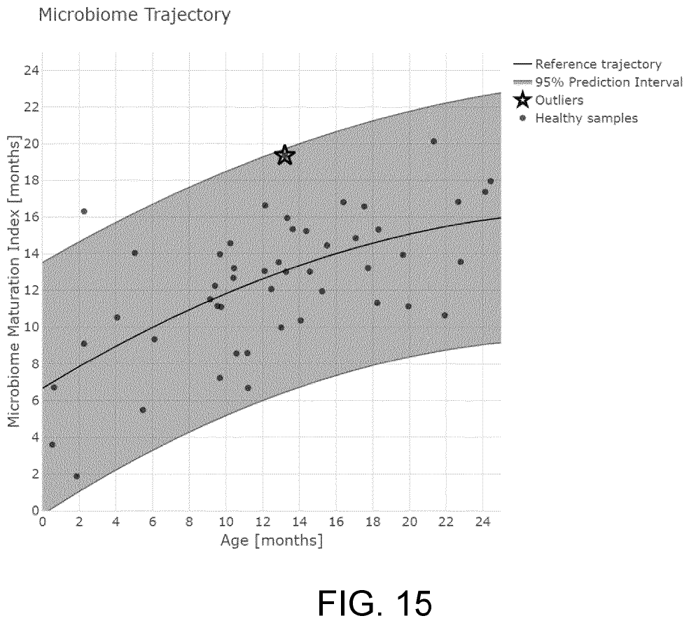

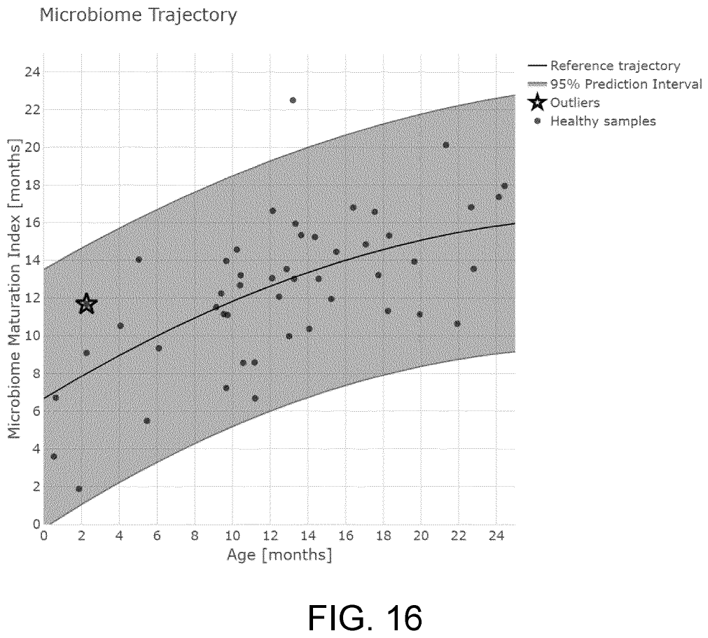

For the off the trajectory example presented in FIG. 9, a similar simulation exercise led to this sample being back on the trajectory as shown in FIG. 13. For the off the trajectory example presented in FIG. 10, a similar simulation exercise led to this sample being back on the trajectory as shown in FIG. 14. For the off the trajectory example presented in FIG. 11, the simulation exercise led to this sample being back on the trajectory as shown in FIG. 15. For the off the trajectory example presented in FIG. 12, the simulation exercise led to this sample being back on the trajectory as shown in FIG. 16.

### Example 6: Personalized Nutrition Interventional Advises to Bring Back Off the Early Life Microbiome (ELM) Trajectory Samples Back on the ELM Trajectory

A similar restoration of the off the trajectory infants back on to the trajectory can be done by personalized food and dietary advises or nutritional supplements such as vitamins and minerals or administration of other prebiotics, probiotics or synbiotics. An example of such advice is shown in Table 2.

TABLE 2
Exemplary nutritional advice to be on the
Early Life Microbiome (ELM) trajectory

| | Nutrient | |
|---|---|---|
| Taxa | requirements | Supporting reference |
| Faecalibacterium | Retinol (Vitamin A) | Michelini, S., et al., 2018. |
| prausnitzii | Acetate | Microbiome, 6(1), pp. 1-18. |
| | Zinc | |
| Faecalibacterium | Riboflavin (Vitamin | Khan, M. T., et al., 2014. |
| prausnitzii | B2) | PLoS One, 9(5), p. e96097. |
| | Cysteine | Khan, M. T., et al., 2012. The |
| | | ISME journal, 6(8), pp. |
| | | 1578-1585. |
| Faecalibacterium | Kestose (Fructo-OS) | Koga, Y., et al., 2016. Pediatric |
| prausnitzii | | research, 80(6), pp. 844-851. |
| Faecalibacterium | Sun dried raisins | Verhoog, S., et al., 2019. |
| prausnitzii | FOS (but possibly | Nutrients, 11(7), p. 1565. |
| | not inulin) | |
| | Polydextrose | |
| | Soluble corn fiber | |
| | Isoflavones with | |
| | FOS | |
| Akkermansia | Pomegranate extract | Verhoog, S., et al., 2019. |
| muciniphila | Resveratrol | Nutrients, 11(7), p. 1565. |
| | Polydextrose | |
| | Yeast fermentate | |
| | Sodium butyrate | |
| | Inulin | |
| Roseburia | Vitamin E | Tang, M., et al., 2016. Journal |
| | | of pediatric gastroenterology and |
| | | nutrition, 63(3), p. 379. |

### Example 7: Statistical Methods

The following formulae were used for defining the fit of the trajectory and determining whether a sample is on or off the ELM trajectory.

### Least Squares Polynomial Fit

$${\{ {\hat{β}}_{j} \}}_{j = 0}^{2} = E$$

$$where E = {\sum }_{i = 0}^{n} \vert y ( x \vert {β}_{0} , {β}_{1} , {β}_{2} ) - {y}_{i} \vert$$

n is the number of samples (xᵢ, yᵢ), i∈[0, n] in the dataset.
xᵢ is independent variable (i.e. age at data collection).
yᵢ is dependent variable to be fitted by model function y with 3 parameters and degree 2 (i.e. Microbiome Maturation Index (MMI)).
β₀, β₁, β₂ are parameters of the model to be optimised to minimise E.

### Chi-Square

First, consider yᵢ has uncertainty described by standard deviation σᵢ. Second, consider that residuals r=y(x|β₀, β₁, β₂)−yᵢ˜N. Then the function:

$${\sum }_{i = 1}^{n} {[ \frac{{y}_{i} - y ( x \vert {β}_{1} … {β}_{m} )}{{σ}_{i}} ]}^{2} ∼ {χ}^{2}$$

Once we find the best parameters for optimal fit, the probability distribution of χ² will be χ² distribution for n−2 degrees of freedom. If y1 has no uncertainty, σᵢ=1. This is only used to measure the goodness of a fit. Moreover, we use reduced chi-square χᵣ² where a good fit should have χᵣ²=1.

### Confidence Interval

The 95% probability interval around a fit line contains the mean of new values at a specific age of collection value. Iterative resampling residuals 500 times, where the darker the colour of overlap, the confidence is even higher.

$$\vert {\hat{μ}}_{y \vert x 0} - {μ}_{y \vert x 0} \vert \leq {T}_{n - 2}^{\text{.975}} \hat{σ} \sqrt{\frac{1}{n} + \frac{{( {x}_{0} - \overline{x} )}^{2}}{{\sum }_{i = 1}^{n} {( {x}_{i} - \overline{x} )}^{2}}}$$

\hat{μ}_{y|x0}=Σᵢ₌₀²aᵢxᵢ is polynomial fit with degree of the polynomial equal to 2.
μ_{y|x0} is the mean response of new values.
x₀ is specific value.
Tₙ₋₂^{0.975} is 97.5^{th} percentile of the Student's t-distribution with n−2 degrees of freedom.
n is number of samples in the dataset.
\hat{σ} is the standard deviation of the error term in the fit:

$$\hat{σ} = \sqrt{{\sum }_{i = 1}^{n} \frac{{( {y}_{i} - \hat{y} )}^{2}}{n - 2}}$$

### Prediction Interval

The 95% probability that this shaded interval around fit line contains a new future observation at specific age at collection value.

$$\vert {\hat{y}}_{0} - {y}_{0} \vert \leq {T}_{n - 2}^{\text{.975}} \hat{σ} \sqrt{1 + \frac{1}{n} + \frac{{( {x}_{0} - \overline{x} )}^{2}}{{Σ}_{i = 1}^{n} {( {x}_{i} - \overline{x} )}^{2}}}$$

ŷ₀Σᵢ₌₀²aᵢx₀ is polynomial fit with degree of the polynomial equal to 2.
y₀ is the new observation.
x₀ is specific value.
Tₙ₋₂^{0.975} is 97.5^{th} percentile of the Student's t-distribution with n−2 degrees of freedom.
n is number of samples in the dataset.
\hat{σ} is the standard deviation of the error term in the fit:

$$\hat{σ} = \sqrt{{\sum }_{i = 1}^{n} \frac{{( {y}_{i} - \hat{y} )}^{2}}{n - 2}}$$

### EMBODIMENTS

Various preferred features and embodiments of the present invention will now be described with reference to the following numbered paragraphs (paras).
1. A method for providing a trained regression model for determining the gut microbiota status of a subject, wherein the method comprises:
   (a) providing gut microbiota data from a population of healthy subjects; and
   (b) training a regression model on the gut microbiota data, wherein the age of the healthy subjects at data collection is regressed on one or more microbial ratios provided from the gut microbiota data.
2. The method according to para 1, wherein the age of the healthy subjects at data collection is regressed on a plurality of microbial ratios provided from the gut microbiota data.
3. The method according to para 1 or 2, wherein the age of the healthy subjects at data collection is regressed on 2 or more microbial ratios, 3 or more microbial ratios, 4 or more microbial ratios, or 5 or more microbial ratios.
4. The method according to any preceding para, wherein the age of the healthy subjects at data collection is regressed on 50 or fewer microbial ratios, 25 or fewer microbial ratios, 10 or fewer microbial ratios, 9 or fewer microbial ratios, or 8 or fewer microbial ratios.
5. The method according to any preceding para, wherein the age of the healthy subjects at data collection is regressed on from 2 to 50 microbial ratios, from 3 to 25 microbial ratios, from 4 to 10 microbial ratios, or from 5 to 8 microbial ratios.
5. The method according to any preceding para, wherein the method further comprises determining the microbial ratios from the gut microbiota data.
6. The method according to any preceding para, wherein the microbial ratios are log-transformed, preferably wherein the logarithm base is 2.
7. The method according to any preceding para, wherein the microbial taxa in the microbial ratios are taxonomically-classified and/or functionally-classified, preferably wherein the microbial taxa in the microbial ratios are taxonomically-classified by phylum, class, order, family, genus and/or species, more preferably wherein the microbial taxa in the microbial ratios are taxonomically-classified by genus.
8. The method according to any preceding para, wherein the microbial ratios are bacterial ratios, preferably wherein the microbial taxa in the microbial ratios comprise one or more bacterial taxa selected from Escherichia, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Bacteroides, Citrobacter, Neisseria, Bifidobacterium, Lachnospira, Dialister, Ruminococcus, Blautia, Streptococcus, Eggerthella, Enterococcus, and Paraprevotella.
9. The method according to any preceding para, wherein the microbial ratios each have the same microbial taxon as the denominator of the ratio.
10. The method according to para 9, the denominator of the ratio is determined by the number of crossings the microbial taxon has with all the other microbial taxa in the dataset and/or on other criteria such as modelling performance statistics, availability or ease of testing, or the subject of interest.
11. The method according to any preceding para, wherein the microbial taxon used as the denominator of the ratio is selected from Escherichia, Bacteroides, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Citrobacter, Neisseria, Bifidobacterium, Lachnospira, Dialister, Ruminococcus, Blautia, Streptococcus, Eggerthella, Enterococcus, and Paraprevotella, preferably wherein the denominator of the ratio is Escherichia or Bacteroides.
12. The method according to any preceding para, wherein the microbial ratios comprise one or more microbial ratio selected from: Roseburia/Escherichia, Faecalibacterium/Escherichia, Sutterella/Escherichia, SMB53/Escherichia, Collinsella/Escherichia, Ruminococcus/Escherichia, Akkermansia/Escherichia, Veillonella/Escherichia, Parabacteroides/Escherichia, Clostridium/Escherichia, Oscillospira/Escherichia, Megasphaera/Escherichia, Fusobacterium/Escherichia, Bacteroides/Escherichia, Citrobacter/Escherichia, Neisseria/Escherichia, and Lachnospira/Escherichia; or wherein the microbial ratios comprise one or more microbial ratio selected from: Roseburia/Bacteroides, Faecalibacterium/Bacteroides, Clostridium/Bacteroides, Bifidobacterium/Bacteroides, Neisseria/Bacteroides, Akkermansia/Bacteroides, Dialister/Bacteroides, Ruminococcus/Bacteroides, Escherichia/Bacteroides, Blautia/Bacteroides, Streptococcus/Bacteroides, Parabacteroides/Bacteroides, Eggerthella/Bacteroides, Collinsella/Bacteroides, Veillonella/Bacteroides, Paraprevotella/Bacteroides, Sutterella/Bacteroides, Enterococcus/Bacteroides, and Megasphaera/Bacteroides.
13. The method according to any preceding para, wherein the trained regression model is an Early Life Microbiome (ELM) trajectory.
14. The method according to any preceding para, wherein the trained regression model predicts the age of a subject given their gut microbiota data.
15. The method according to any preceding para, wherein the trained regression model relates the age of a healthy subject to their microbiota age, microbiome maturation index, and/or microbiome maturation age, preferably wherein the microbiota age is a microbiota compositional age and/or a microbiota functional age.
16. The method according to any preceding para, wherein the trained regression model is for infancy and/or early childhood, preferably wherein the trained regression model is for 0-5 years of age, 0-3 years of age, 0-2 years of age, more preferably wherein the trained regression model is for 0-24 months of age.
17. The method according to any preceding para, wherein the trained regression model is for 0-12 months of age, 6-12 months of age, or 12-24 months of age.
18. The method according to any preceding para, wherein the method further comprises obtaining the gut microbiota data from the population of healthy subjects.
19. The method according to any preceding para, wherein the gut microbiota data is obtained from or obtainable from fecal samples.
20. The method according to any preceding para, wherein the gut microbiota data provides the relative abundance and/or absolute abundance for a plurality of microbial taxa, preferably wherein the gut microbiota data provides the relative abundance for a plurality of microbial taxa.
21. The method according to any preceding para, wherein the gut microbiota data is obtained or obtainable by PCR-based detection, semi-quantitative detection methods, cycling temperature capillary electrophoresis, immunological-based methods, or any combination thereof.
22. The method according to any preceding para, wherein the gut microbiota data from a population of healthy subjects comprises at least 40 samples.
23. The method according to any preceding para, wherein the healthy subjects are infants and/or children, preferably wherein the healthy subjects are 0-5 years of age, or 0-3 years of age, or 0-2 years of age, more preferably wherein the healthy subjects are 0-24 months of age.
24. The method according to any preceding para, wherein the healthy subjects are 0-12 months of age, 6-12 months of age, or 12-24 months of age.
25. The method according to any preceding para, wherein the population of healthy subjects comprises at least 20 healthy subjects.
26. The method according to any preceding para, wherein the regression model is a tree-based regression model, preferably a random forest regression model.
27. The method according to any preceding para, wherein the age of the healthy subjects at sample collection is also regressed on one or more additional features provided from the gut microbiota data.
28. A trained regression model obtained or obtainable by a method according to any of paras 1 to 27.
29. A trained regression model for determining the gut microbiota status of a subject given one or more microbial ratios provided from the subject's gut microbiota data.
30. The trained regression model according to para 29, wherein the trained regression model is obtained or is obtainable by a method according to any of paras 1 to 27.
31. A method for predicting the age of a subject, wherein the method comprises:
   (a) providing a trained regression model by a method according to any of paras 1 to 27, or a trained regression model according to any of paras 28-30;
   (b) providing gut microbiota data from the subject; and
   (c) predicting the age of the subject given their gut microbiota data and the trained regression model.
32. A method for determining the gut microbiota status of a subject, wherein the method comprises:
   (a) providing a trained regression model by a method according to any of paras 1 to 27, or a trained regression model according to any of paras 28-30;
   (b) providing gut microbiota data from the subject; and
   (c) determining whether the subject is an outlier or not in the trained regression model;
      wherein the gut microbiota status of the subject is healthy if the subject is not an outlier in the trained regression model, and/or wherein the gut microbiota status of the subject is not healthy if the subject is an outlier in the trained regression model.
33. The method according to para 32, wherein the subject is an outlier based on the standard errors (SE), confidence intervals, prediction intervals, and/or standard deviations in the trained regression model, preferably wherein the subject is an outlier if their gut microbiota data is −2SE or less or 2SE or more from the trained regression line, if their gut microbiota data falls outside the 95% confidence interval in the trained regression model, if their gut microbiota data falls outside the 95% prediction interval in the trained regression model, and/or if they have a Z-score of −2 or less or 2 or more in the trained regression model, more preferably wherein the subject is an outlier if their gut microbiota data falls outside the 95% prediction interval in the trained regression model.
34. A method for determining the gut microbiota status of a subject, wherein the method comprises:
   (a) providing a trained regression model by a method according to any of paras 1 to 27, or a trained regression model according to any of paras 28-30, wherein the trained regression model is an ELM trajectory;
   (b) providing gut microbiota data from the subject; and
   (c) determining whether the subject is on or off the ELM trajectory;
      wherein the gut microbiota status of the subject is healthy if the subject is on the ELM trajectory, and/or wherein the gut microbiota status of the subject is not healthy if the subject is off the ELM trajectory.
35. The method according to para 34, wherein the subject is on the ELM trajectory if the subject's gut microbiota data does not differ significantly from the ELM trajectory and/or wherein the subject is off the ELM trajectory if the subject's gut microbiota data differs significantly from the ELM trajectory.
36. The method according to para 34 or 35, wherein the subject is determined to be off the ELM trajectory based on the standard errors (SE), confidence intervals, prediction intervals, and/or standard deviations of the ELM trajectory, preferably the subject is determined to be off the ELM trajectory if their gut microbiota data is −2SE or less or 2SE or more from the ELM trajectory, if their gut microbiota data falls outside the 95% confidence interval of the ELM trajectory, if their gut microbiota data falls outside the 95% prediction interval of the ELM trajectory, and/or if they have a Z-score of −2 or less or 2 or more, more preferably wherein the subject is determined to be off the ELM trajectory if their gut microbiota data falls outside the 95% prediction interval of the ELM trajectory.
37. A method for determining the gut microbiota status of a subject, wherein the method comprises predicting the age of the subject by a method according to para 31, and wherein the gut microbiota status of the subject is healthy if the predicted age of the subject does not differ significantly from the actual age of the subject and/or wherein the gut microbiota status of the subject is not healthy if the predicted age of the subject differs significantly from the actual age of the subject.
38. The method according to para 37, wherein the predicted age of the subject differs significantly from the actual age of the subject if the predicted age of the subject differs significantly from the actual age of the subject if it differs by about 0.1 years or more, by about 0.2 years or more, by about 0.3 years or more, by about 0.4 years or more, by about 0.5 years or more, by about 0.6 years or more, by about 0.7 years or more, by about 0.8 years or more, by about 0.9 years or more, or by about 1 year or more.
39. The method according to any of paras 31 to 38, wherein the subject's gut microbiota data is obtained or obtainable by PCR-based detection, semi-quantitative detection methods, cycling temperature capillary electrophoresis, immunological-based methods, or any combination thereof.
40. The method according to any of paras 31 to 39, wherein the subject's gut microbiota data provides 2 or more microbial ratios, 3 or more microbial ratios, 4 or more microbial ratios, or 5 or more microbial ratios.
41. The method according to any of paras 31 to 40, wherein the subject's gut microbiota data provide 50 or fewer microbial ratios, 25 or fewer microbial ratios, 10 or fewer microbial ratios, 9 or fewer microbial ratios, 8 or fewer microbial ratios, or 7 or fewer microbial ratios.
42. The method according to any of paras 31 to 41, wherein the subject's gut microbiota data provides from 2 to 50 microbial ratios, from 3 to 25 microbial ratios, from 4 to 10 microbial ratios, or from 5 to 7 microbial ratios.
43. A method for maintaining or improving the gut microbiota status of a subject, wherein the method comprises:
   (a) determining the gut microbiota status of the subject by a method according to any of paras 32 to 42; and
   (b) adjusting the diet, nutrient intake, and/or lifestyle of the subject to maintain or improve the subject's gut microbiota status.
44. The method according to para 43, wherein after adjusting the diet, nutrient intake, and/or lifestyle of the subject the gut microbiota status of the subject is healthy.
45. The method according to para 43 or 44, wherein the adjusted diet, nutrient intake, and/or lifestyle of the subject increases the abundance and/or function of favourable microbial taxa and/or to decreases the abundance and/or function of unfavourable microbial taxa.
46. A method for determining a subject's diet and/or nutrient intake, wherein the method comprises:
   (a) determining the gut microbiota status of the subject by a method according to any of paras 32 to 42; and
   (b) determining the diet and/or nutrient intake required to maintain or improve the gut microbiota status of the subject.
47. The method according to any of paras 43 to 46, wherein the subject is administered food and/or supplements to increase the abundance and/or function of favourable microbial taxa and/or to decrease the abundance and/or function of unfavourable microbial taxa.
48. The method according to para 47, wherein the food and/or supplements comprise prebiotics, probiotics, synbiotics, vitamins, such as Riboflavin (Vitamin B2), Retinol (Vitamin A), and Calciferol (Vitamin D), and/or minerals, such as Manganese, Zinc, and Potassium.
49. A computer program comprising instructions which, when the program is executed by a computer, cause the computer to carry out the method according to any of paras 1 to 27 or 31 to 42.
50. A computer program comprising instructions which, when the program is executed by a computer, cause the computer to determine a trained regression model for determining the gut microbiota status of a subject from a population of healthy subjects, given the age of the healthy subjects at data collection and one or more microbial ratios provided from the healthy subject's gut microbiota data.
51. A computer program comprising instructions which, when the program is executed by a computer, cause the computer to predict the age of a subject or determine the gut microbiota status of a subject, given one or more microbial ratios provided from the subject's gut microbiota data.
52. A computer program comprising instructions which, when the program is executed by a computer, cause the computer to predict the age of a subject or determine the gut microbiota status of a subject, given a regression model trained on the gut microbiota data from a population of healthy subjects and the subject's gut microbiota data, wherein the regression model was trained by regressing the age of the healthy subjects at data collection on one or more microbial ratios provided from the healthy subject's gut microbiota data.
53. The computer program according to para 52, wherein the trained regression model is a trained regression model according to any of paras 28-30.
54. A computer-readable medium comprising instructions which, when executed by a computer, cause the computer to carry out the method according to any of paras 1 to 27 or 31 to 42.
55. A computer-readable medium comprising instructions which, when executed by a computer, cause the computer to determine a trained regression model for determining the gut microbiota status of a subject from a population of healthy subjects, given the age of the healthy subjects at data collection and one or more microbial ratios provided from the healthy subject's gut microbiota data.
56. A computer-readable medium comprising instructions which, when the program is executed by a computer, cause the computer to predict the age of a subject or determine the gut microbiota status of a subject, given one or more microbial ratios provided from the subject's gut microbiota data.
57. A computer-readable medium comprising instructions which, when executed by a computer, cause the computer to predict the age of a subject or determine the gut microbiota status of a subject, given a regression model trained on the gut microbiota data from a population of healthy subjects and the subject's gut microbiota data, wherein the regression model was trained by regressing the age of the healthy subjects at data collection on one or more microbial ratios provided from the healthy subject's gut microbiota data.
58. The computer-readable medium according to para 57, wherein the trained regression model is a trained regression model according to any of paras 28-30.
59. Use of one or more microbial ratios provided from a subject's gut microbiota data to predict the age of the subject or to determine the gut microbiota status of the subject.
60. Use of one or more microbial ratios provided from gut microbiota data from a population of healthy subjects to train a regression model.
61. Use of a trained regression model according to any of paras 28-30 to predict the age of a subject or to determine the gut microbiota status of a subject.
62. The method according to any of paras 31-48, the computer program according to any of paras 49-53, the computer-readable medium according to any of paras 54-58, or the use according to any of paras 59-61, wherein the subject is an infant or a child, preferably wherein the subject is 0-5 years of age, or 0-3 years of age, or 0-2 years of age, more preferably wherein the subject is 0-24 months of age.
63. The method according to any of paras 31-48, the computer program according to any of paras 49-53, the computer-readable medium according to any of paras 54-58, or the use according to any of paras 59-61, wherein the subject is 0-12 months of age, 6-12 months of age, or 12-24 months of age.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for providing a trained regression model for determining the gut microbiota status of a subject, wherein the method comprises:
(a) providing gut microbiota data from a population of healthy subjects; and
(b) training a regression model on the gut microbiota data, wherein the age of the healthy subjects at data collection is regressed on one or more microbial ratios provided from the gut microbiota data.

2. The method according to claim 1, wherein the microbial ratios are bacterial ratios.

3. The method according to claim 1, wherein the microbial ratios each have the same microbial taxon as the denominator of the ratio.

4. The method according to claim 1, wherein the method further comprises obtaining the gut microbiota data from the population of healthy subjects.

5. The method according to claim 1, wherein the healthy subjects are infants and/or children.

6-7. (canceled)

8. A method for determining the gut microbiota status of a subject, wherein the method comprises:
(a) providing a trained regression model by a method comprising providing gut microbiota data from a population of healthy subjects; and training a regression model on the gut microbiota data, wherein the age of the healthy subjects at data collection is regressed on one or more microbial ratios provided from the gut microbiota data;
(b) providing gut microbiota data from the subject; and
(c) determining whether the subject is an outlier or not in the trained regression model;
wherein the gut microbiota status of the subject is healthy if the subject is not an outlier in the trained regression model, and/or wherein the gut microbiota status of the subject is not healthy if the subject is an outlier in the trained regression model.

9. The method according to claim 8, wherein the subject's gut microbiota data is obtained or obtainable by a method selected from the group consisting of PCR-based detection, semi-quantitative detection methods, cycling temperature capillary electrophoresis, immunological-based methods, and any combination thereof.

10. A method for maintaining or improving the gut microbiota status of a subject, wherein the method comprises:
(a) determining the gut microbiota status of the subject comprising providing a trained regression model by a method comprising providing gut microbiota data from a population of healthy subjects; and training a regression model on the gut microbiota data, wherein the age of the healthy subjects at data collection is regressed on one or more microbial ratios provided from the gut microbiota data; providing gut microbiota data from the subject; and determining whether the subject is an outlier or not in the trained regression model; wherein the gut microbiota status of the subject is healthy if the subject is not an outlier in the trained regression model, and/or wherein the gut microbiota status of the subject is not healthy if the subject is an outlier in the trained regression model; and
(b) adjusting the diet, nutrient intake, and/or lifestyle of the subject to maintain or improve the subject's gut microbiota status.

11. The method according to claim 10, wherein the subject is administered food and/or supplements to increase the abundance and function of favourable microbial taxa.

12-15. (canceled)
